(19) United States
(12) Patent Application Publication
(10) Pub. No.: US 2014/0057284 A1
(43) Pub. Date: Feb. 27, 2014

(54) PHOTOLUMINESCENT MOLECULAR COMPLEX AND METHOD FOR DETERMINING OF THE CONCENTRATION OF SAID MOLECULAR COMPLEX

(71) Applicants: Asko Uri, Tartu (EE); Erki Enkvist, Tartu (EE); Angela Vaasa, Tartu County (EE); Marje Kasari, Tartu (EE)

(72) Inventors: Asko Uri, Tartu (EE); Erki Enkvist, Tartu (EE); Angela Vaasa, Tartu County (EE); Marje Kasari, Tartu (EE)

(73) Assignee: UNIVERSITY OF TARTU, Tartu (EE)

(21) Appl. No.: 13/982,165

(22) PCT Filed: Jan. 26, 2012
(86) PCT No.: PCT/EP12/51275

§ 371 Date: Nov. 12, 2013

(30) Foreign Application Priority Data
Jan. 26, 2011 (EP) 11152289.2

(51) Int. Cl. C12Q 1/48

(52) U.S. Cl. CPC C12Q 1/485
USPC 435/7.2; 435/188; 435/15

(57) ABSTRACT

The invention concerns novel molecular complexes with photoluminescent probes whose specific association with purine-binding proteins leads to increased emission of long lifetime luminescence, and the application of the probes for monitoring activity of protein kinases (PKs) and other purine-binding proteins, screening of compounds as inhibitors of PKs and characterization of inhibitors targeted to the kinase, and methods of manufacturing of such probes. The invention concerns also the use of the improved method for monitoring activity of protein kinases in living cells, characterization of inhibitors of protein kinases, analysis of protein kinase-based disease biomarkers and other tasks of biological and medical importance.

### TECHNICAL FIELD

The invention concerns novel molecular complexes with photoluminescent probes whose specific association with purine-binding proteins leads to increased emission of long lifetime luminescence, and the application of the probes for monitoring activity of protein kinases (PKs) and other purine-binding proteins, screening of compounds as inhibitors of PKs and characterization of inhibitors targeted to the kinase, and methods of manufacturing of such probes.

The invention also relates to the application of the luminescent complexes in biochemical and cellular screening assays for identifying compounds that bind to and modulate the activity of a wide variety of members of PK super-family. The invention relates to the assay for identification and evaluation of PK inhibitors and determination of the concentration of PKs.

Molecular complexes of the invention can be used for, for example, monitoring the responsiveness of cells to receptor activation, high throughput screening of inhibitors of PKs and agonists and antagonists of metabotropic and ionotropic receptors, medical diagnosis, tissue imaging.

### BACKGROUND ART

Altogether ca 3000 proteins (protein kinases, small G proteins, dehydrogenases, ATPases, helicases, non-conventional purine-utilizing proteins, synthtases, purinergic receptors, etc.) are known to possess a purine-binding site, forming the humane purinome. Thus, approximately 13% of protein-coding genes of the human genome are devoted to coding for purine-binding proteins. [Knapp et al., Curr. Top. Med. Chem. 6 (2006) 1129] PKs are the most prevalent of proteins binding a purine nucleotide, ATP. The human genome codes 518 different PKs. [Manning et al., Science 298 (2002) 1912]

Protein Kinases and Protein Kinase Inhibitors

PKs play a key role in the regulation of protein functions in living cells. The activity of one third of proteins is regulated through phosphorylation of one or more of serine, theorine and thyrosine residues of the protein catalyzed by a PK. More than 400 human diseases (incl. cancer) have been linked to aberrant PK signaling. This has made PK the second largest drug target (and fastest growing category of drugs in development) after G protein-coupled receptors. [Cohen, Nat. Rev. Drug Discov. 1 (2002) 309; Fischer, Curr. Med. Chem. 11 (2004) 1563; Zhang et al., Nat Rev Cancer. 9 (2009) 28] Almost 150 inhibitors targeted to 40 different PKs have reached clinical trials. [Fedorov, Nature Chem. Biol. 6 (2010) 166]

PKs follow ternary complex kinetic mechanism in which direct transfer of the phosphoryl group from ATP to the protein substrate occurs in the active site. [Adams, Chem. Rev. 101 (2001) 2271]

The majority of known inhibitors realize their inhibitory potency via competitive with ATP binding to the purine-binding pocket of PKs. Despite serious selectivity problems (all 518 PKs and more than 2500 other proteins possess a purine-binding site) the main efforts of drug companies have been directed to the development of small-molecule ATP-competitive inhibitors.

Bisubstrate-analog (biligand) inhibitors of PKs also compete with ATP for binding to the PK. These inhibitors additionally associate with the protein binding domain of the enzyme. [Lavogina et al., ChemMedChem 5 (2010) 23]

Existing Binding Assays for PKs

Development of new assay methods for monitoring the activity of PKs and evaluation of inhibitors of PKs has run in parallel with the increase of importance of effective inhibitors for drug industry. [Zaman et al., Comb Chem High Throughput Screen, 6 (2003) 313]

Photoluminescence methods can be spatially and temporally more focused than radioactive methods, and as such, they are more suitable for application in high-throughput screening (HTS) assays. [Olive, Expert Rev Proteomics. 1 (2004) 327]

Some small-molecule inhibitors can be conjugated to fluorescent dyes leading to luminescent probes (indicators) that bind to PKs. The term “luminescent probe” as used herein is a compound whose optical (luminescent) properties are changed on binding with a target protein. Usually, the emission intensity, anisotropy and/or lifetime are altered when the analyte (PK) is bound to the probe. The probe can then be displaced by a competitive kinase inhibitor and the change in luminescence characteristics measured to establish the affinity of the non-labeled inhibitor. This interaction therefore forms the basis for a competitive binding assay of kinase inhibitors where neither a substrate nor an antibody to the phosphorylated substrate is required. Several papers and patent applications describe the use of fluorescent probes for determination of the binding characteristics of PK inhibitors. [e.g., Chen et al., 268 (1993) 15812, WO2005/033330].

The importance of binding assays for PKs is quickly increasing [Uri et al., Biochimica et Biophysica Acta 1804 (2010) 541] as small-molecular fluorescent probes can be used for both biochemical assays in kinase solutions and for measurements in living cells.

Limitations of Current Binding Assays

Both binding and optical properties of luminescent probes have limited their use in binding assays of PKs. Fluorescent probes with micromolar affinity towards PKC were disclosed by Chen et al. [Chen et al., 268 (1993) 15812]. Low (micromolar) affinity and complex fluorescence spectrum make it difficult to use these fluorescent conjugates as probes for binding assays. [WO9906590] Another fluorescent probe described in the patent application WO2005/033330 has affinity in high nanomolar range (K_{d}=161 nM) which leads to the need for high concentration of the kinase in the assay format (200 nM STK12 kinase was used in the disclosed Example). The requested kinase concentration, arising from the high K_{d} value of the fluorescent probe, is a hundred-fold higher than the kinase concentration usually applied for kinetic measurements (ca 1 nM concentration of the enzyme is often used in kinetic assays). This leads to substantial increase of the cost of the assay. Due to sub-micromolar dissociation constant of the probe it cannot be used for determination of binding constants of inhibitors with nanomolar affinity [Fluorescence Polarization Technical Resource Guide THIRD EDITION 2004, Invitrogen Corporation. Chapter 7]

Bisubstrate inhibitor-based fluorescent probes for PK binding assays with steady-state fluorescence polarization/anisotropy, fluorescence intensity and FRET detection have also been described previously. [WO2008019696; Vaasa et al., Anal Biochem 385 (2009) 85; Uri et al., Biochimica et Biophysica Acta 1804 (2010) 541]

The application of long lifetime luminescent labels enables the use of time-gated measurements where the signal is measured with certain delay time after the excitation pulse. This leads to the increase of signal/background ratio. Time-resolved (TR) luminometry (time resolution in micro- or millisecond range) is an excellent measuring regime for homogenous assays because it can free the measured signal from nanosecond-lifetime background fluorescence caused by organic compounds and light scattering. Several recent publications have described the application TR-FRET phenomenon for detection of binding of fluorescent probes to PKs. [Zhang et al., Analytical Biochemistry 343 (2005) 76; Lebakken et al., J Biomol Screen 14 (2009) 924; Kwan et al., Analytical Biochemistry 395 (2009) 256; WO 2010/127980] All these applications use antibodies or other recognition proteins (e.g., avidine, streptavidine) labeled with lanthanide cryptates (or chelates) in combination with fluorescently labeled small-molecule PK inhibitors to get a measurable TR-FRET signal for the complex. In addition to the complexness (both fluorescent probes and antibodies labelled with lanthanide complexes are needed, slow binding of antibodies to the antigen takes time to complete, etc.) and high price of such detection systems, problems with stability of lanthanide complexes in cellular milieu and inability of antibodies to penetrate cell plasma membranes and their instability in cellular milieu make the use of such systems non-applicable for measurements in living cells.

Room-Temperature Long Lifetime Luminescence of Small Organic Molecules

Room-temperature phosphorescence of aromatic compounds in water solution occurs in special conditions. Cyclodextrin-enhanced room-temperature phosphorescence is known for many compounds. [Scypinski et al., Anal. Chem. 56 (1984) 331] Tryptophan phosphorescence of proteins at room temperature has been used as a tool to study protein structure and dynamics [Papp et al., Photochem. Photobiol. 49 (1989) 775] Room temperature phosphorescence of some aromatic compounds in the liquid state is also a powerful tool in analytical chemistry. [Kuijt et al., Anal. Chim. Acta 488 (2003) 135]

Förster-type of non-radiative dipole-dipole energy transfer [Förster, Ann. Physik. 6 (1948) 55] takes place between two molecules in condition where their energies (emission of donor D with absorption of acceptor A) overlap and they are located at a suitable distance from each other. In addition to the more usual transfer from short lifetime singlet state, radiationless intermolecular resonance energy transfer from long lifetime triplet state of the donor to the singlet state of the acceptor is also in agreement with the Forster dipole-dipole interaction and this phenomen has also been described. [Bennett et al., J. Chem. Phys. 41 (1964) 3040] These data point to the possibility of application of long lifetime luminescent labels as donors for FRET measurements in combination with short lifetime fluorescence acceptors. The substantial enhancement of emission induced by RET from a long-lifetime, low quantum yield donor based on metal-ligand complexes to a long-wavelength, high quantum yield acceptor, has been described and explained. [WO2002/007779; Kang et al., J. Fluoresc. 12 (2002) 97] This phenomenon was visible both inter- and intramolecularly when a ruthenium complex (metal-ligand complex, MLC) with microsecond luminescence lifetime was used as a donor in this system, and a fluorescent red dye BO-PRO-3 was used as the fluorescence acceptor excited by resonance energy transfer. [Maliwal et al., Anal. Chem. 73 (2001) 4277]

All previously described methods based on TR-FRET phenomenon use binding proteins labeled with lanthanide cryptates (or chelates) in combination with fluorescently labeled small-molecule PK inhibitors to get a TR-FRET signal for the complex. This make these assays expensive and binding of antibodyes require long incubation times. Problems with the stability of metal-ligand complexes in cellular milieu and the inability of antibodies to penetrate cell plasma membranes make the use of such expensive systems non-applicable for measurements in living cells.

Benefits of the Current Invention

The current invention concerns complexes with small-molecule optical probes whose specific binding to purine-binding site of the target protein leads to substantial change of parameters (e.g., intensity and lifetime) of long lifetime (1-1000 microseconds) luminescence.

The molecular complexes of the current invention are applicable in binding assays for determination of the concentration (activity) of PKs and identification and characterization of inhibitors of PKs. If compared to the probes from previous inventions, the luminescence probes of the present invention are small organic molecules that in complex with a purine-binding protein emit long wavelength light with long lifetime (in microsecond region) that enables the time-gated discrimination over fluorescence present in the sample, either autofluorescence of cells or emission from other fluorophores. A single small-molecule synthetic compound is needed to perform the analysis. The luminescence of the probes in a measurement window after longer delay time (more than 10 microseconds from the excitation pulse) is substantially increased upon specific binding of the luminescence donor into the purine-binding pocket of the target protein. The novel luminescent probes contain no volumous and degradable metal-ligand complexes that have been previously used as luminescence donors for TR-FRET measurements. The end-point assays based on the use of luminescent probes of the current invention are very quick, sensitive, and require the use of only one additional chemical (luminescent probe). This makes the assays amenable to automatization and very cost-effective.

### DISCLOSURE OF INVENTION

The invention relates to molecular complexes comprising small-molecule photoluminescent probes a long lifetime luminescence energy donor and a fluorescence energy acceptor for use in binding assays with purine-binding proteins. The non-radiative energy transfer based emission of the acceptor is detected in a suitable time window after delay from donor excitation pulse. The specific binding of the purine-mimicking fragment of the probe to a PK leads to substantial change in the intensity and lifetime of the emitted light. The concentration of the probe bound to a PK in the sample is determined based on the change of the intensity of the emitted light. The complexes possess a great gap between the excitation and measurement wavelengths.

The invention also concerns biochemical and cellular bioaffinity assays and tissue imaging with time-resolved luminescence readout based on the use of the molecular complexes.

### BRIEF DESCRIPTION OF DRAWINGS

FIG. 1. Scheme of the kinase induced long lifetime luminescence. LP-long-pass filter, T-luminescence lifetime.

FIG. 2. Scheme of the kinase induced long lifetime luminescence in the case of intermolecular resonance energy transfer. LP-long-pass filter T-luminescence lifetime.

FIG. 3. General structure of a long-lifetime luminescent probe.

FIG. 4. Titration of the luminescent probe ARC-1063 (5 nM) with PKs MSK1 (), ROCKII (♦), AKT3 (▴), PKA (▪), PKG (▾), or PKC (x).

FIG. 5. Titration of the luminescent probe ARC-1139 (2 nM) with PKs MSK1 (), ROCKII (♦), AKT3 (▴), PKA (▪), PKG (▾), or PKC (x).

FIG. 6. Displacement of the luminescent probe ARC-1063 [at the concentration of 54 nM (▪), 18 nM (▴), 6 nM (▾), or 2 nM (♦)] from its complex with PKAc by ARC-902. In the inset the Schild plot analysis of the data is presented (▪).

FIG. 7. Displacement of the luminescent probe ARC-1063 (10 nM) from its complex with ROCK (1 nM) by Fasudil (), Y27632 (▾), or Staurosporine (▴).

FIG. 8. Correlation between the values of inhibition constants (Kᵢ) measured in TLC kinetic inhibition assay and displacement constants (K_{d}) determined in binding assay using the probe ARC-1063.

FIG. 9. Titration of the labelled kinase TAMRA-PKAc with ARC-668. An example of enhancement of luminescence by intermolecular RET.

FIG. 10. Increase of long lifetime luminescence intensity that results from the binding of ARC-1063 to the kinase dissociated from complex with ARC-583(Δ), ARC-1041(▪), or Staurosporine(). Examples of the measurement of the kinetics of dissociation of the inhibitor from its complex with the kinase.

FIG. 11. Normalized emission intensity at 675 nm in living C9H6 cells in the presence (▪) or absence (□) of ARC-1139. Treatment of the cells with Forskolin (FRSK) leads to dissociation of PKAc from the holoenzyme resulting in the interaction of PKAc with ARC-1139 that leads to increased emission at 675 nm (excitation at 337 nm). The latter effect was reversed by a cell-permeable PKA inhibitor H89.

FIG. 12. Activation of PKA by adrenergic agonist isoproterenol in live HEK293 cells as measured through the change of luminescence from ARC-1139.

FIG. 13. Activation of PKA by Forskolin in C9H6 cells as measured with ARC-1139 (25 nM) in the cell lysate.

FIG. 14. Measurement of low concentrations of the kinase PKAc in human plasma. The luminescence signal of 2 nM ARC-1063 in human blood plasma spiked with concentration range of recombinant PKAc (0-333 pM) in the absence (filled symbols) and presence (empty symbols) of both ATP (100 μM) and PKA selective inhibitor PKI (100 μM). The luminescence signal is directly proportional to the amount of PKA present in the sample. Moreover, the concentration of the endogeneous PKA in blood plasma can be determined from the difference of the results obtained in the presence and absence of PKI. Extracellular PKAc in blood plasma is an acknowledged cancer biomarker. [WO/2000/079281]

### BEST MODE FOR CARRYING OUT THE INVENTION

The present invention relates to molecular complexes, which comprise a heteroaromatic moiety serving as a long-lifetime luminescence (phosphorescence) donor bound to the purine-binding pocket of the protein and a fluorescent luminescence acceptor dye, which exhibits photoluminescence properties of long lifetime after irradiation.

In particular, the invention relates to molecular complexes, which comprise an active purine-binding protein, a non-metal long-lifetime luminescence (phosphorescence) donor with heteroaromatic moiety binding to the purine-binding pocket of the protein and a fluorescent luminescence acceptor dye with excitation spectrum, which is at least partially overlapping with the luminescence emission spectrum of the luminescence donor, wherein the molecular complex possesses long lifetime (1-1000 microseconds) photoluminescence properties, if the complex is irradiated at excitation wavelengths of said luminescence donor and the luminescence is measured at emission wavelengths of said fluorescent luminescence acceptor dye.

The invention is related to this kind of molecular complex, wherein the luminescence donor with heteroaromatic moiety and fluorescent luminescence acceptor dye are covalently connected by an organic linker.

Moreover, the fluorescent luminescence acceptor dye of the molecular complex is covalently connected to the purine-binding protein.

The luminescence donor of the molecular complex of the invention contains a thiophene, selenophene or a tellurophene moiety.

More particularly, the purine-binding protein of the molecular complex is a protein kinase.

In a preferred embodiment of the invention, the compound incorporating the luminescence donor in the molecular complex of the invention is a bisubstrate inhibitor of a protein kinase.

The purine-binding protein of said molecular complex may be a protein kinase labelled with a fluorescent luminescence acceptor dye or a protein kinase fused with a fluorescent protein having excitation and emission properties of said fluorescent luminescence acceptor dye.

The invention also provides a method for determining of the concentration of the said molecular complex, wherein the complex is excited with a flash of light in the wavelength range 200-450 nM and the intensity of the emitted light is measured in the wavelength range 450-1000 nm after 0.1-500 microsecond delay time.

The said method can be carried out, if the complex is excited at the wavelengths above 400 nm in the format of multiphoton excitation and the complex emits light in the wavelength range 450-1000 nm.

Thus, another object of the invention is a method for screening and characterization of protein kinase inhibitors, wherein the reduction of the concentration of a molecular complex of the invention is determined in the presence of inhibitory compounds.

Also, a method is provided for determining binding and dissociation rate constants for inhibitors binding to a purine-binding protein by monitoring the concentration of the molecular complex.

A binding assay is provided for monitoring the concentration of active protein kinases in living cells based on the determination of the concentration of a molecular complex of the invention, wherein the luminescence of the cells is measured with a luminescence microscope, a luminescence spectrometer or a luminescence plate reader with pulse excitation and time delayed (time-resolved) luminescence detection possibilities.

This kind of binding assay can be effectively used for in vivo characterization of inhibitors of protein kinases.

The method provided in the invention can be used for characterization of agonists and antagonists of Gs and Gi protein-coupled receptors through measuring the change in luminescence intensity caused by the activation of PKA by the second messenger cAMP in living cells.

Moreover, the method provided in the invention can be used for in vivo characterization of agonists and antagonists of metabotropic and ionotropic receptors by measuring the change in the concentration of molecular complexes of the invention caused by the activation of protein kinases other than PKA.

Similarly, the method provided in the invention can be used for determination of the concentration of active protein kinases in body fluids, cell lysates and tissue extracts.

As another embodiment, the method provided in the invention can be used for monitoring of receptor activation and cellular concentration of cAMP by analysing cell lysates.

Yet another embodiment of the invention enables determination of the concentration of active protein kinases in body fluids, blood cells, and tissue extracts as biomarkers of diseases.

A method of the invention can also be used for in vivo application for mapping of protein kinase activity in cells, tissues and organisms.

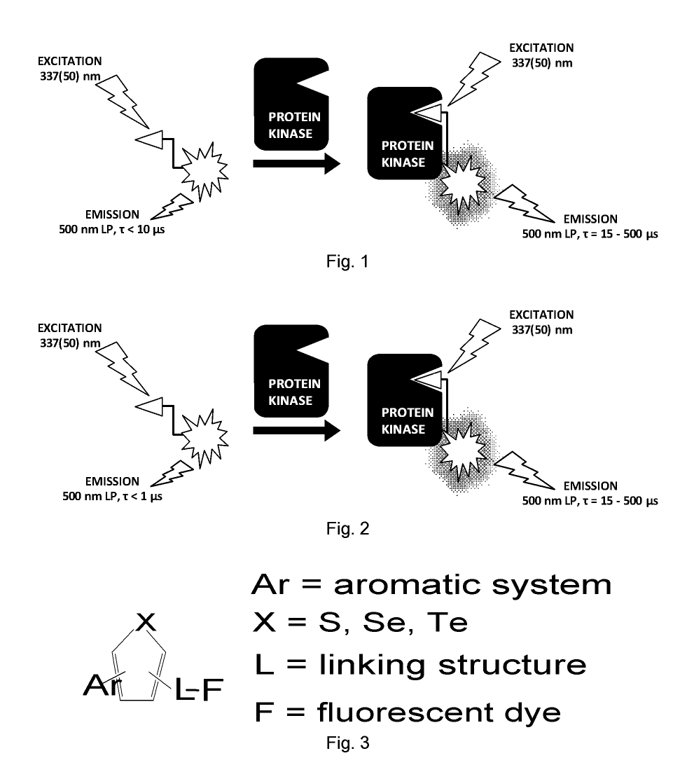

The molecular complexes of the invention involve long-lifetime photoluminescence probes, whose binding to the target protein induces substantial alteration of the signal intensity emitted by the complex (FIG. 1).

We established and improved molecular structures of luminescent probes whose specific association with the purine-binding site of the target protein leads to substantial change in long lifetime photoluminescence. Our attempt to develop small-molecule long lifetime photoluminescent probes for PKs was based on the following previous knowledge.
1. Phosphorescence of flexible aromatic molecules which are restricted in their excited state reorientation by fixation into a complex with a binding molecule (e.g., cyclodextrin) is largely increased, such binding also reduces collisional quenching of luminescence by oxygen and other impurities.
2. Heavy atoms in aromatic systems have been shown to increase phosphorescence of organic compounds.
3. The possibility of substantial enhancement of emission induced by RET from a long-lifetime, low quantum yield donor to a long-wavelength, high quantum yield acceptor has been previously been shown.

Some organic compounds have shown significant room temperature phosphorescence in micelles, cyclodextrin complexes, polymeric matrixes and crystals. Room temperature phosphorescence originating from the tryptophan residues of a protein has been used to probe structure and molecular mobility of proteins. Restriction of molecular movements is required to diminish nonradiative relaxations of triplet states in all cases of room temperature phosphorescence of pure organic luminophors to produce a detectable signal. Enhancement of luminescence through resonance energy transfer from emitters with low quantum yield to bright energy acceptors has been described. [WO2002/007779]

The current invention provides molecular complexes involving compounds with the formula P-L-F, wherein P contains a fragment incorporating a long lifetime luminescence donor that binds specifically to a purine-binding pocket of the target protein leading to substantial alteration of the lifetime and intensity of the emitted light when the donor is excited, L is a linker system, and F is a fluorescent dye with good light absorbance and high quantum yield. The most important roles that the linker L has to play in the structure of the luminescent probe P-L-F is the increase of binding affinity of the structural fragment P, tuning of the kinase selectivity of P towards specific PKs, the positioning of the fluorescent label F in a location where it causes minimal hindrance to the binding of the luminescent probe to the kinase, at the same time F should be positioned close to P to achieve maximal energy transfer from the luminescence donor P to the acceptor F. In the case of ATP-competitive inhibitors L may possess a fragment of acknowledged inhibitors whose role in the starting inhibitor is to influence the affinity and selectivity profile of the inhibitor towards PK-s and other purine-binding proteins. P incorporates a fragment of the inhibitor whose optical properties are strongly influenced by its binding to the purine-binding site of the target protein.

The first embodiment of the invention is a complex with a luminescent probe comprising a long-lifetime luminescence donor and a fluorescence acceptor.

More particularly, the probes in the molecular complex of the present invention have the structure

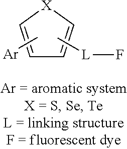

where the aromatic system Ar contains differently substituted (hetero)aromatic rings (FIG. 3).

An important unit of the luminescent probes is the heteroaromatic moiety

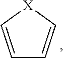

containing as the atom X a sulphur (S, in the case of thiophene), selenium (Se, in the case of selenophene) or tellurium (Te, in the case of tellurophene) atom. Other 5-membered S-, Se- or Te-containing heterocycles, e.g., these that are additionally incorporating 1-4 nitrogen atoms, also possess optical characteristics supporting their use as optical donor moieties of luminescent probes of the present invention. These 5-membered S-, Se- or Te-containing heterocycles may be also substituted with halogeno groups and other functional groups.

The aromatic system Ar incorporates structural units whose role is the probe is the enhancement of binding affinity of the probe to the target protein and the tuning of the binding selectivity towards purine-binding proteins. Ar also tunes the optical properties of the probe making P a long life-time luminescence donor of the probe and rendering the luminescence of the probe sensitive to its binding to the protein. The Ar moiety may be connected to a single atom of the thiophene (selenophene, tellurophene) ring or simultaneously fused to two atoms of the ring while retaining the conjugation between Ar moiety and the thiophene (selenophene, tellurophene) ring. Aromatic systems tested include six-membered heteroaromatic rings and purine-type fused heteroaromatic systems (Table 1) but are not limited to these disclosed in the Table 1 as inhibitory potency of many other thiophene-containing aromatic systems towards different PKs has been previously reported.

Table 1. ARC-codes, structures and HRMS data of the compounds (deconvoluted monoisotopic mass is presented)

TABLE 1

| ARC- | | HRMS |
|---|---|---|
| code | Structure | Found |
| ARC- 668 | 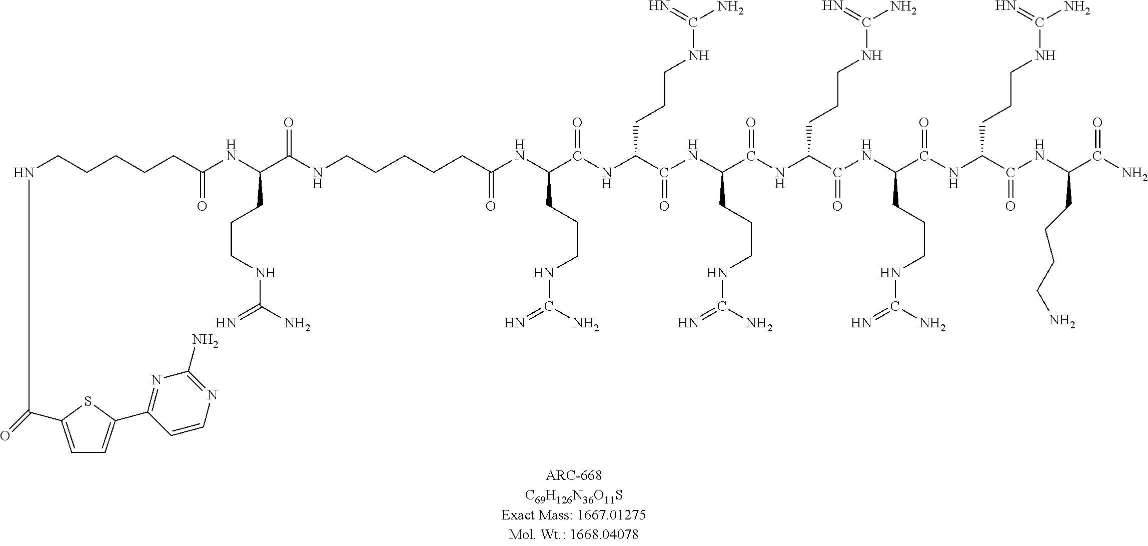 [SMILES: N=C(N)NCCC[C@@H](NC(=O)CCCCCNC(=O)c1ccc(-c2ccnc(N)n2)s1)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCN)C(N)=O] | 1667.01219 |
| ARC- 669 | 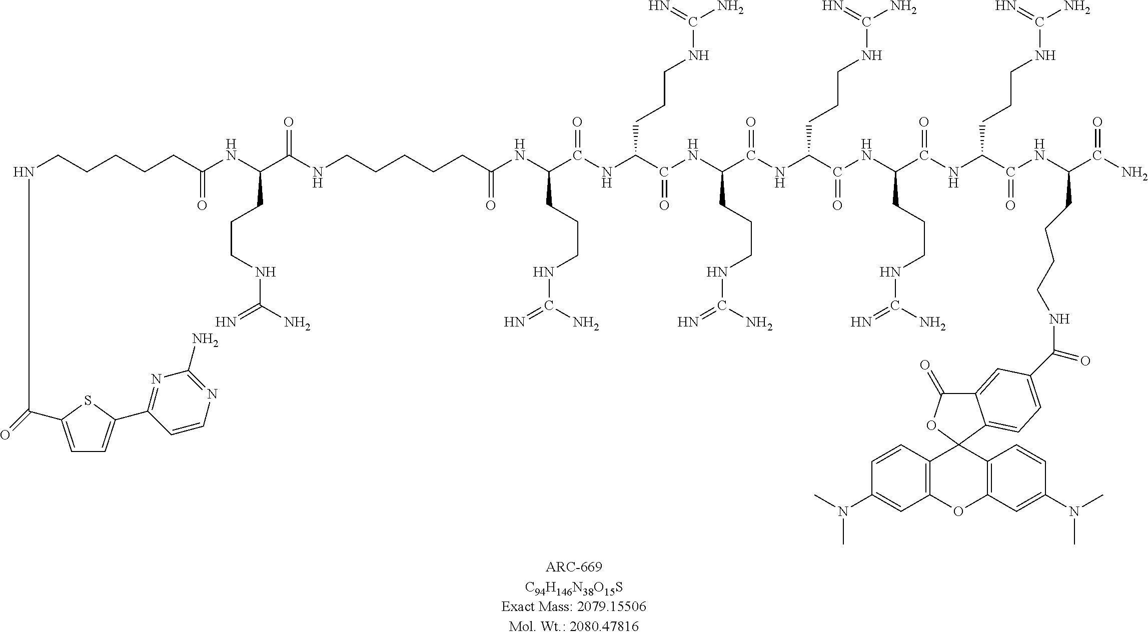 [SMILES: CN(C)c1ccc2c(c1)Oc1cc(N(C)C)ccc1C21OC(=O)c2cc(C(=O)NCCCC[C@@H](NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)CCCCCNC(=O)[C@@H](CCCNC(=N)N)NC(=O)CCCCCNC(=O)c3ccc(-c4ccnc(N)n4)s3)C(N)=O)ccc21] | 2079.15284 |
| ARC- 676 | 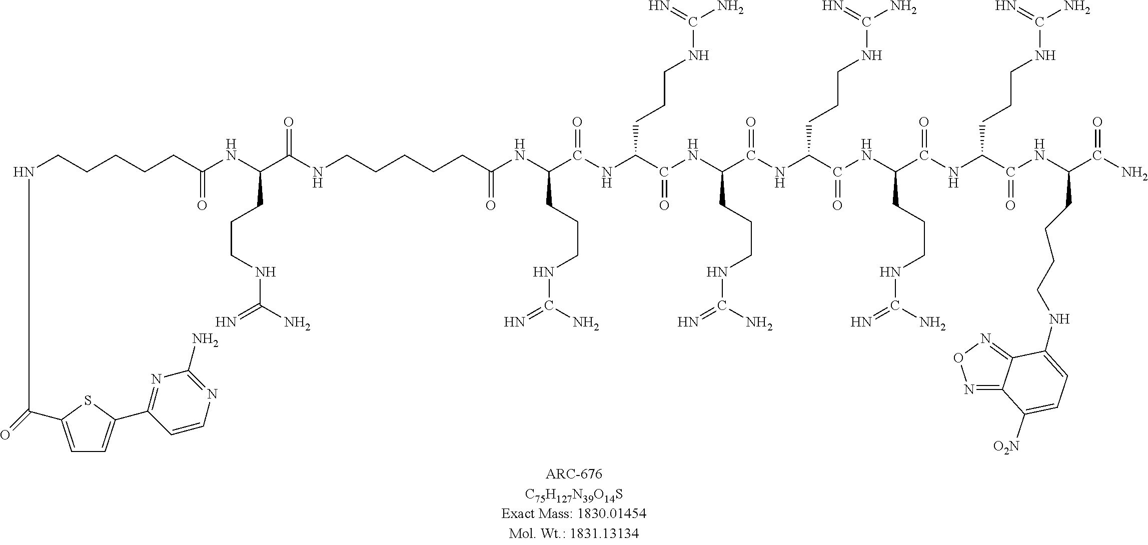 [SMILES: N=C(N)NCCC[C@@H](NC(=O)CCCCCNC(=O)c1ccc(-c2ccnc(N)n2)s1)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCNc1ccc([N+](=O)[O-])c2nonc12)C(N)=O] | 1830.01281 |
| ARC- 679 | 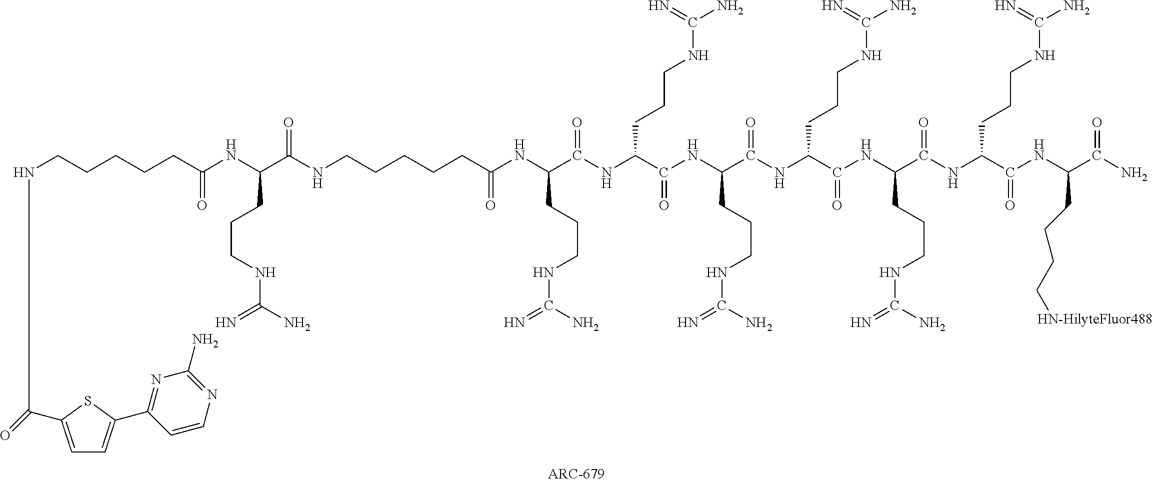 | 2136.1739* |
| ARC- 1063 | 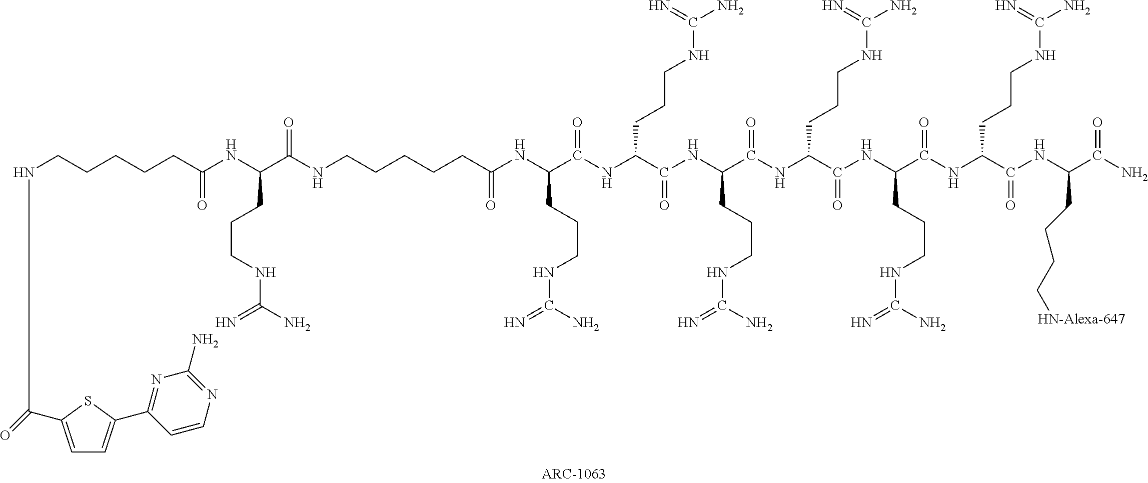 | 2507.1846* |
| ARC- 1121 | 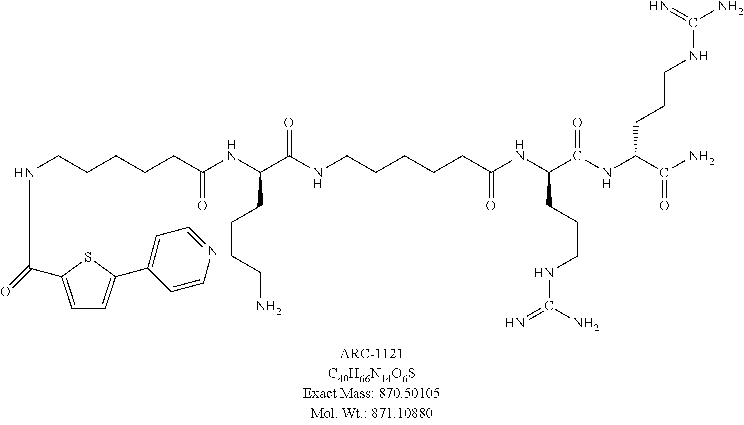 [SMILES: N=C(N)NCCC[C@@H](NC(=O)[C@@H](CCCNC(=N)N)NC(=O)CCCCCNC(=O)[C@@H](CCCCN)NC(=O)CCCCCNC(=O)c1ccc(-c2ccncc2)s1)C(N)=O] | 870.49849 |
| ARC- 1122 | 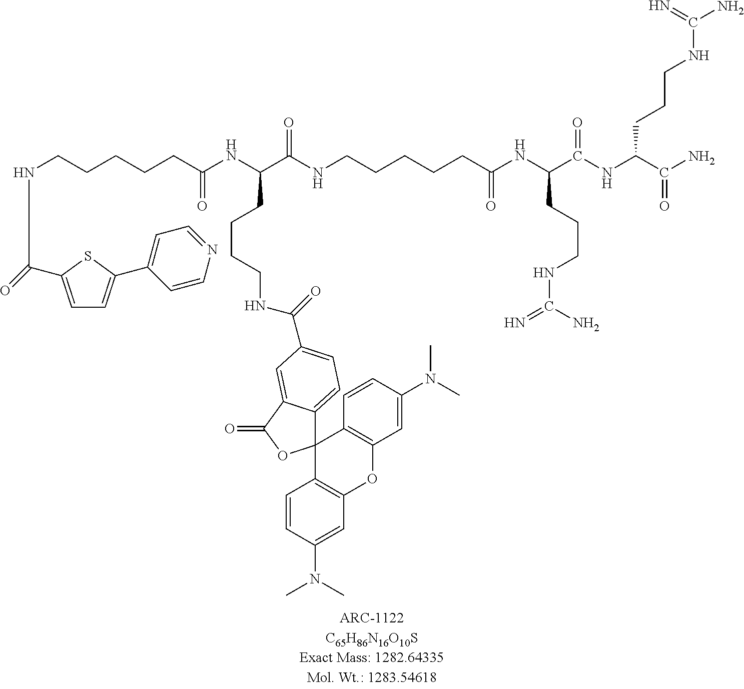 [SMILES: CN(C)c1ccc2c(c1)Oc1cc(N(C)C)ccc1C21OC(=O)c2cc(C(=O)NCCCC[C@@H](NC(=O)CCCCCNC(=O)c3ccc(-c4ccncc4)s3)C(=O)NCCCCCC(=O)N[C@H](CCCNC(=N)N)C(=O)N[C@H](CCCNC(=N)N)C(N)=O)ccc21] | 1282.64083 |
| ARC- 1123 | 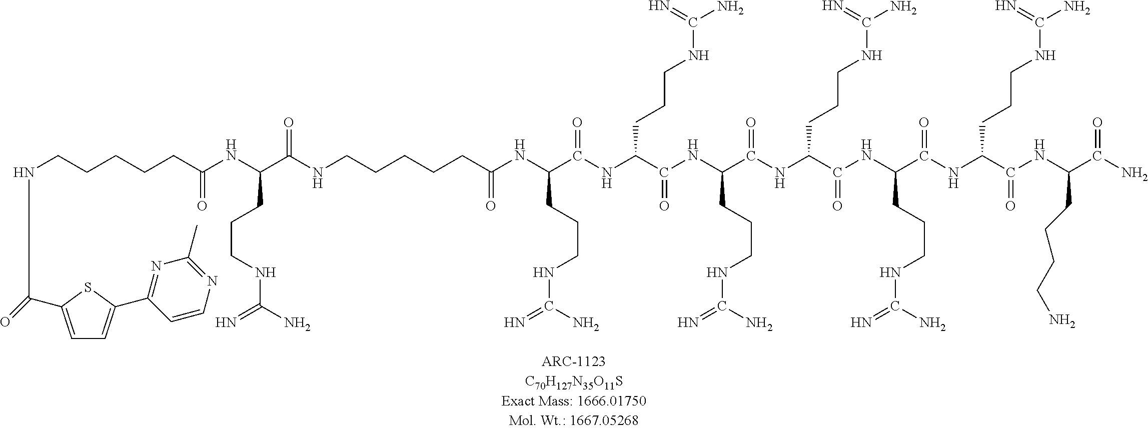 [SMILES: Cc1nccc(-c2ccc(C(=O)NCCCCCC(=O)N[C@H](CCCNC(=N)N)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCN)C(N)=O)s2)n1] | 1666.01168 |
| ARC- 1128 | 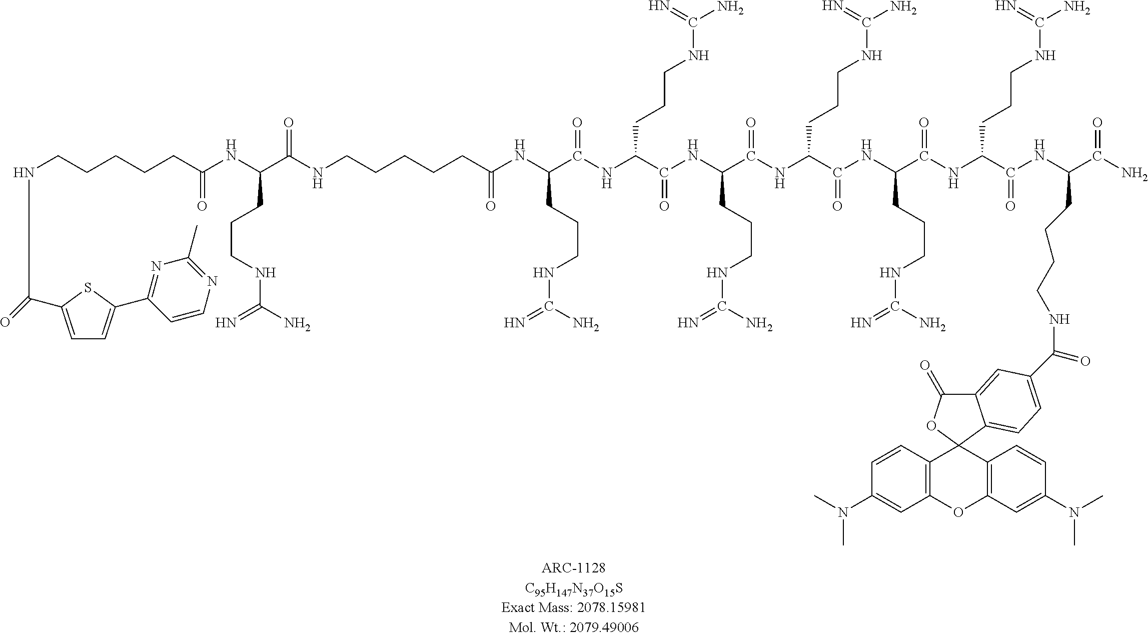 [SMILES: Cc1nccc(-c2ccc(C(=O)NCCCCCC(=O)N[C@H](CCCNC(=N)N)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCNC(=O)c3ccc4c(c3)C(=O)OC43c4ccc(N(C)C)cc4Oc4cc(N(C)C)ccc43)C(N)=O)s2)n1] | 2078.16026 |
| ARC- 1129 | 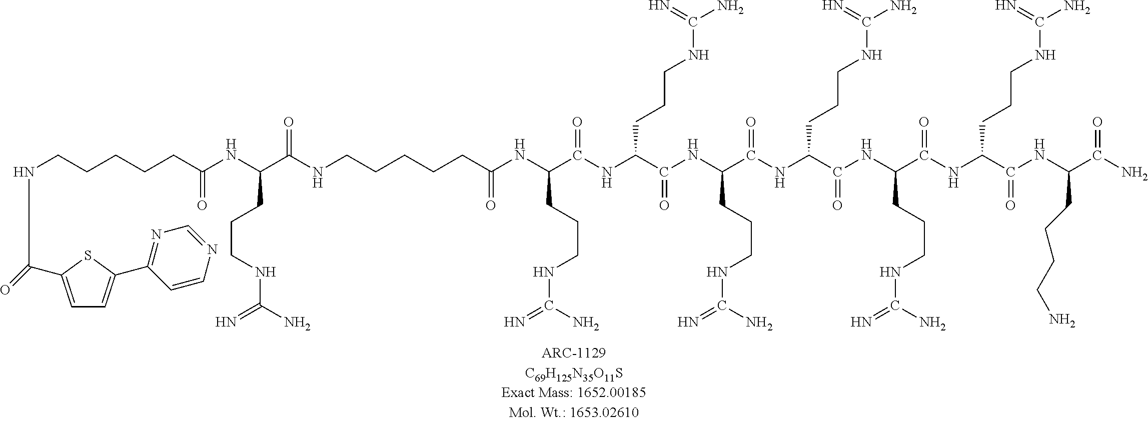 [SMILES: N=C(N)NCCC[C@@H](NC(=O)CCCCCNC(=O)c1ccc(-c2ccncn2)s1)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCN)C(N)=O] | 1651.99576 |
| ARC- 1130 | 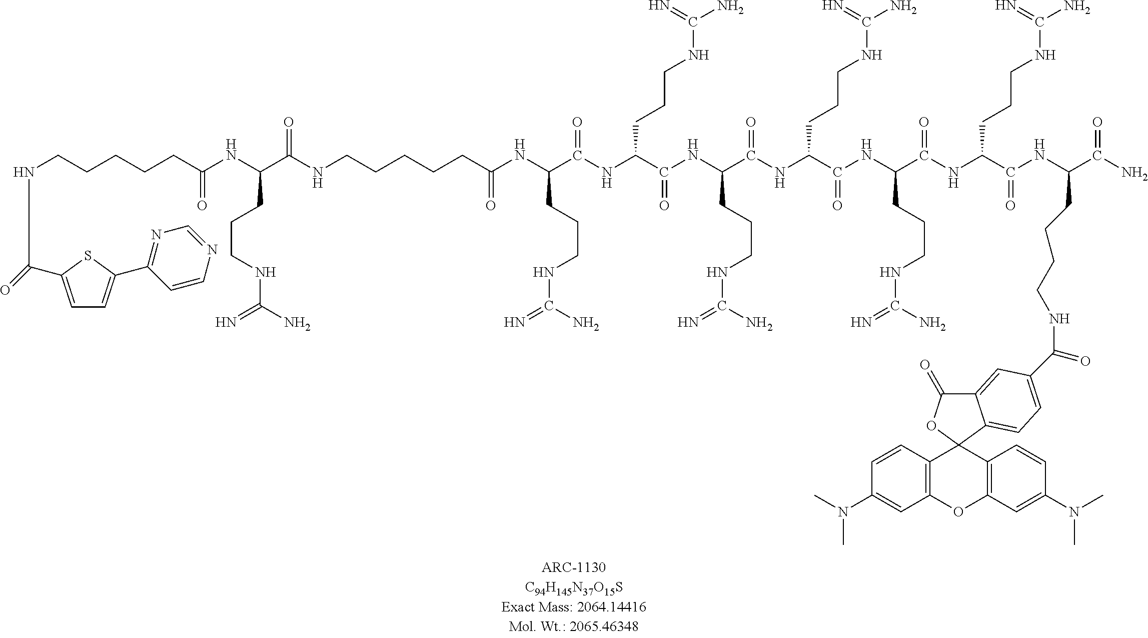 [SMILES: CN(C)c1ccc2c(c1)Oc1cc(N(C)C)ccc1C21OC(=O)c2cc(C(=O)NCCCC[C@@H](NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)CCCCCNC(=O)[C@@H](CCCNC(=N)N)NC(=O)CCCCCNC(=O)c3ccc(-c4ccncn4)s3)C(N)=O)ccc21] | 2064.14487 |
| ARC- 1132 | 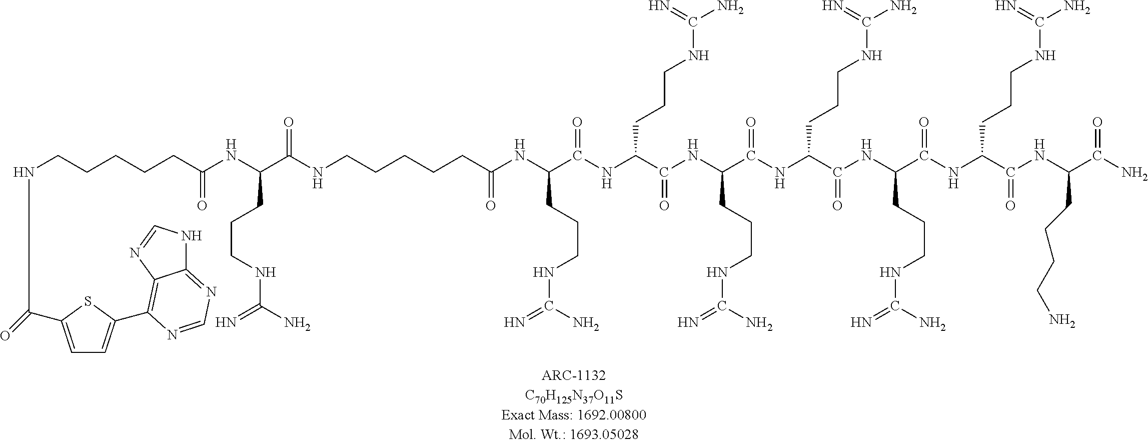 [SMILES: N=C(N)NCCC[C@@H](NC(=O)CCCCCNC(=O)c1ccc(-c2ncnc3[nH]cnc23)s1)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCN)C(N)=O] | 1692.01165 |
| ARC- 1133 | 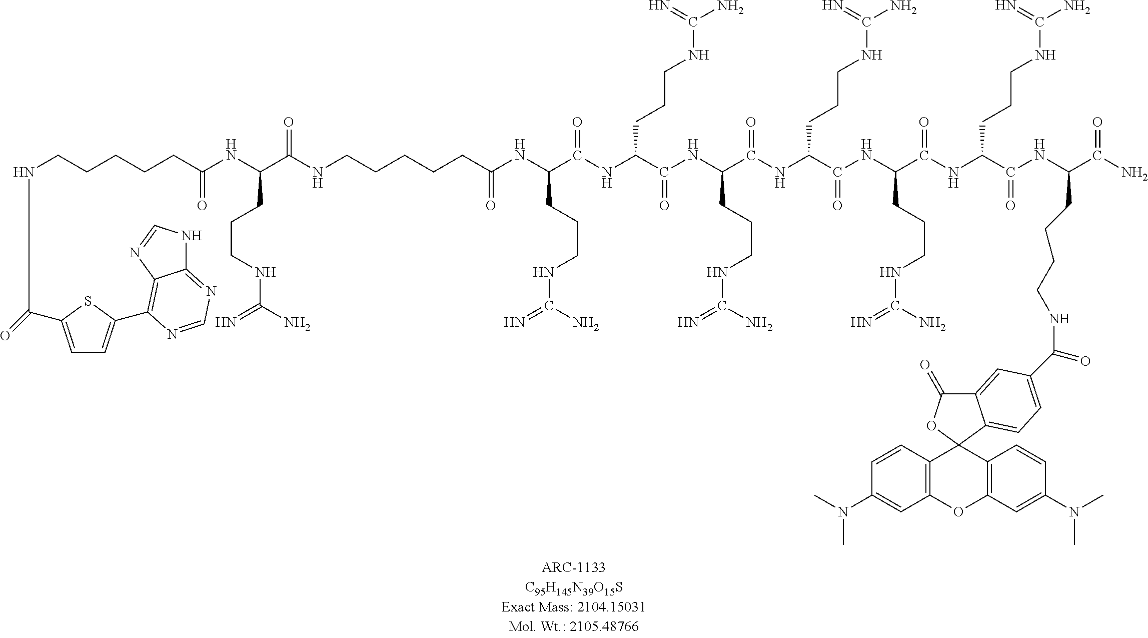 [SMILES: CN(C)c1ccc2c(c1)Oc1cc(N(C)C)ccc1C21OC(=O)c2cc(C(=O)NCCCC[C@@H](NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)CCCCCNC(=O)[C@@H](CCCNC(=N)N)NC(=O)CCCCCNC(=O)c3ccc(-c4ncnc5[nH]cnc45)s3)C(N)=O)ccc21] | 2104.15625 |
| ARC- 1135 | 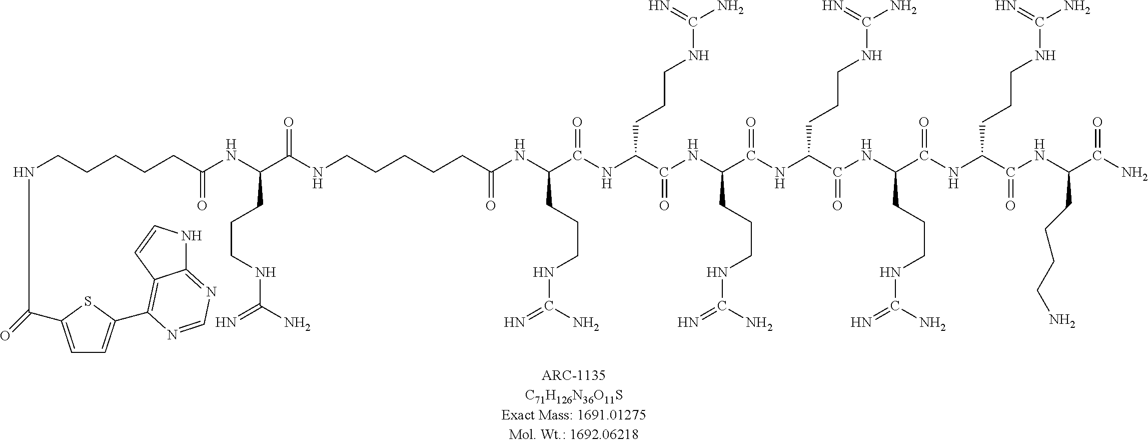 [SMILES: N=C(N)NCCC[C@@H](NC(=O)CCCCCNC(=O)c1ccc(-c2ncnc3[nH]ccc23)s1)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCN)C(N)=O] | 1691 |
| ARC- 1136 | 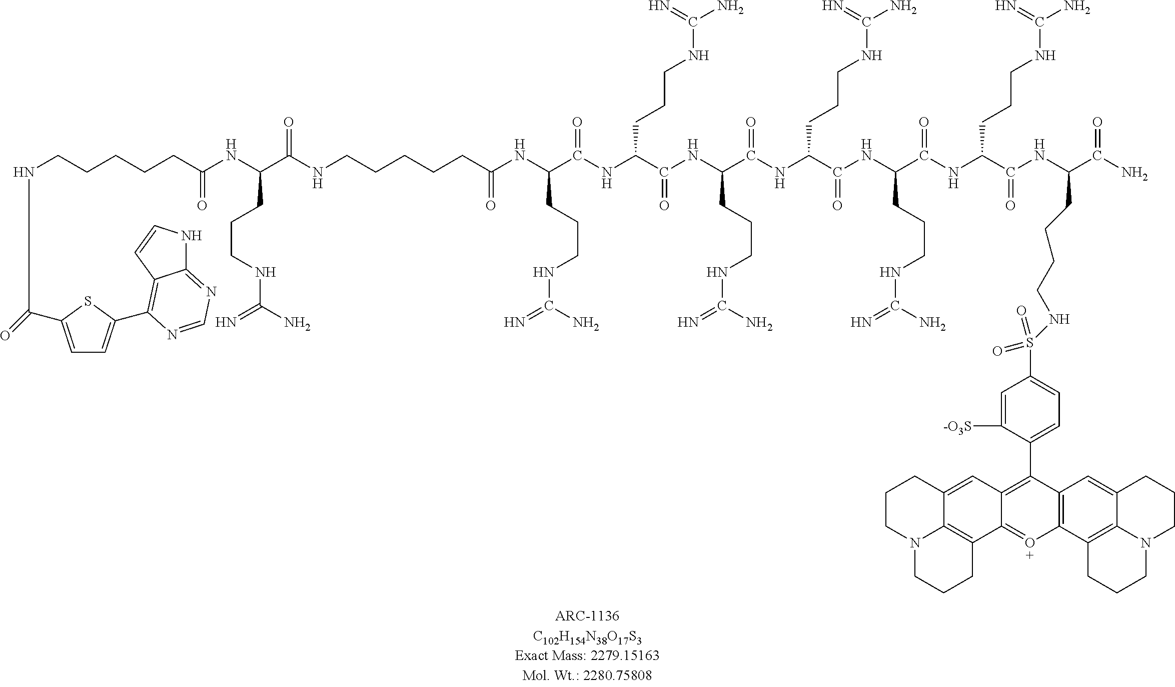 | 2279 |
| ARC- 1138 | 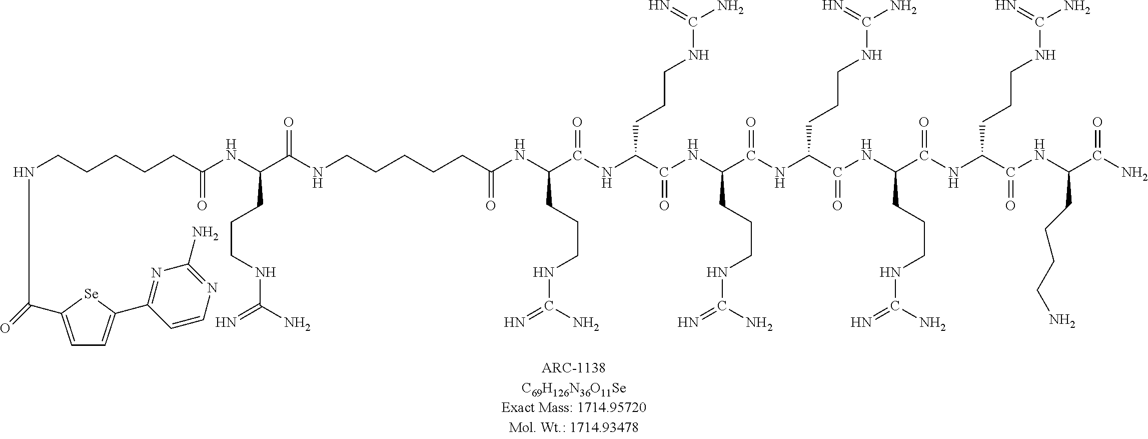 [SMILES: N=C(N)NCCC[C@@H](NC(=O)CCCCCNC(=O)c1ccc(-c2ccnc(N)n2)[se]1)C(=O)NCCCCCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCCN)C(N)=O] | 1714.95391 |
| ARC- 1139 | 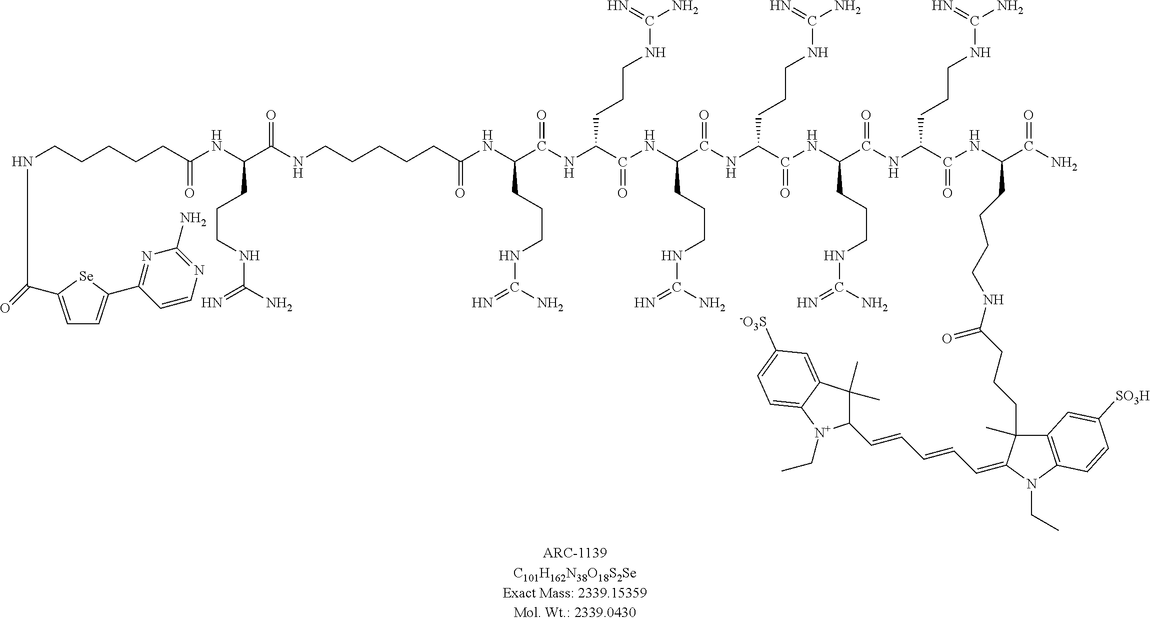 | 2339.14580 |
| ARC- 1144 | 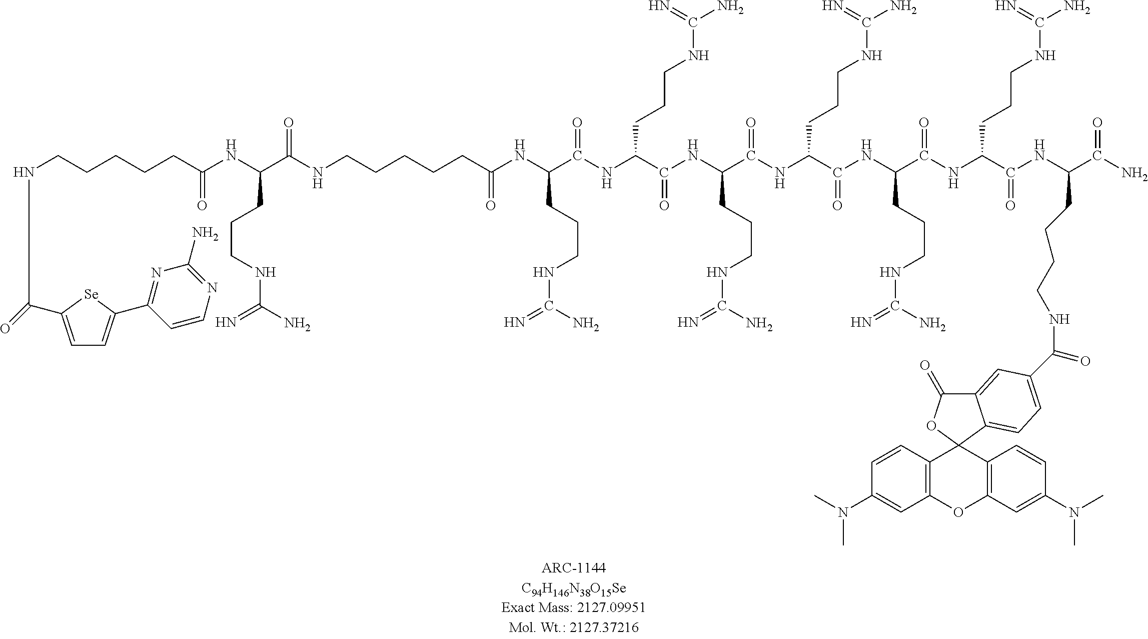 [SMILES: CN(C)c1ccc2c(c1)Oc1cc(N(C)C)ccc1C21OC(=O)c2cc(C(=O)NCCCC[C@@H](NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)CCCCCNC(=O)[C@@H](CCCNC(=N)N)NC(=O)CCCCCNC(=O)c3ccc(-c4ccnc(N)n4)[se]3)C(N)=O)ccc21] | 2127.09949 |

*Structures of HilyteFluor448 and Alexa-647 have not been disclosed.
Monoisotopic masses for selenium containing compounds are calculated for most abundant isotope ⁸⁰Se.

Structures of several compounds incorporating P and L moieties but lacking the fluorescent label F are also presented in Table 1 (compounds ARC-668, ARC-676, ARC-1121, ARC-1123, ARC-1129, ARC-1132, ARC-1135, ARC-1138). These compounds with high affinity (subnanomolar or low nanomolar) bind to the catalytic subunit of protein kinase A (PKAc) and some other PKs of the AGC group. They strongly absorb light between 280-360 nm (molar absorption coefficients 10000-25000 M⁻¹ cm⁻¹) and when excited at 337(50) nm show week long lifetime luminescence (phosphorescence) in the 450-600 nm region in bound to the kinases form (fluorescence spectrometer LS 55 from Perkin Elmer and luminescence platereader PHERAStar fom BMG were used were measurements throughout this work).

The fluorescent acceptor F of the luminescent probe in the molecular complex of the current invention should exhibit a large extinction coefficient and a good quantum yield, and its excitation spectrum should overlap with the emission spectrum of the long lifetime luminescence donor P. Fluorescence labels whose excitation spectrum better overlaps with the emission spectrum of the luminescence donor, who have longer emission wavelengths, and greater photostability are preferred, but the choice of the label also depends on the origin of the analytical instrument used for the assay and special analytical situation. Fluorescent dyes that may be useful in the present invention include inter alia fluorescein and fluorescein derivatives, rhodamine and rhodamine derivatives, cyanine dyes and many other fluorescent labels that are produced and sold by different companies (e.g., Invitrogen, PromoCell, ATTO-TEC GmbH, GE Heathcare, Sigma-Aldrich). Other fluorescent labels that absorb light in the 400-900 nm range and emit light in the 450-1000 nm range are suitable for the application as the fluorescent acceptor moiety F of the luminescence probes of the present invention. Well-known fluorescence dyes FITC, TAMRA Cy3, Cy3B, Cy5, Bodipy 564/570, Texas Red, Alexa Fluor 647, PromoFluor 647 and others have shown suitable optical characteristics when used in combination with thiophene and selenophene-based fragments P. Fluorescent probes incorporating mineral fluorescent labels, e.g., quantum dots, may be useful for special applications. Luminescent probes P-L-F (e.g., compounds ARC-669, ARC-676, ARC-679, ARC-1063, ARC-1122, ARC-1128, ARC-1130, ARC-1133, ARC-1136, ARC-1139, and ARC-1144 in Table 1) are excited at wavelengths below 400 nm with single-photon excitation and at wavelengths over 600 nm when multiphoton excitation is used. The emitted radiation is measured at wavelengths 450-1000 nm

5-carboxytetramethylrhodamine (5-TAMRA) is a good fluorescent label to be used as a fluorescence acceptor of a luminescence probe of the current invention, as the dye is a single-position isomer, the dye is exited at a suitable wavelength, it has high brightness and great photostability.

Many other fluorescent labels are suitable for the application as the acceptor moiety F of the luminescence probes. These labels are described in the Handbook: [The Handbook—A Guide to Fluorescent Probes and Labeling Technologies Web Edition of The Handbook, Tenth Edition 2006 Invitrogen Corporation]

In the case of other luminescent probes the linker unit L may be a C₁-C₂₀ alkylene group, a C₁-C₂₀ alkenylene group, a C₁-C₂₀ alkynylene group, wherein one or more of the CH₂ groups present in the C₁-C₂₀ alkylene group, C₁-C₂₀ alkenylene group, C₁-C₂₀ alkynylene group is optionally replaced with —O—, —C(O)—, —C(O)N—, —S—, —S(O)—, —SO₂—, —N(R)—; R is H or C₁₋₆ alkyl. In the case of some probes L may be omitted.

The novel fluorescently labelled thiophene- or selenophene-based luminescent probes possess 15-500 microseconds lifetimes when these probes are bound to the purine-binding site of a PK (FIG. 1). Binding of the probes to different kinases leads to a luminescence signal whose magnitude is dependent on the origin of the probe and that of the kinase (FIG. 5). When longer delay times (e.g., >50 microseconds) after the excitation pulse are used for the measurement of the emission signal, the binding of the probes to the target PKs leads to 50-2500-fold increase of the luminescence signal. Microsecond lifetimes of the probes enable the performance of time gated measurements with the application of commercial platereaders and microscopes.

Another embodiment of the present invention is the use of a PK inhibitor possessing a long lifetime luminescence donor properties (P-L) but lacking a fluorescent dye, e.g., ARC-668 (Table 1), in combination with a PK that is labeled with an organic dye, fused with a fluorescence protein or bound to specific fluorescently labelled antibody. Other tagging methods (e.g., FlAsH, HaloTag, etc.) can be use for labelling of the target proteins. The binding of the probe P-L to the labelled kinase that leads to intermolecular resonance energy transfer from the bound luminescence donor to the labelled protein (FIG. 2) that can be detected measuring the signal emitted by the fluorescence acceptor.

A special embodiment of the invention is the complex comprising the luminescent probe ARC-669 with the structure:

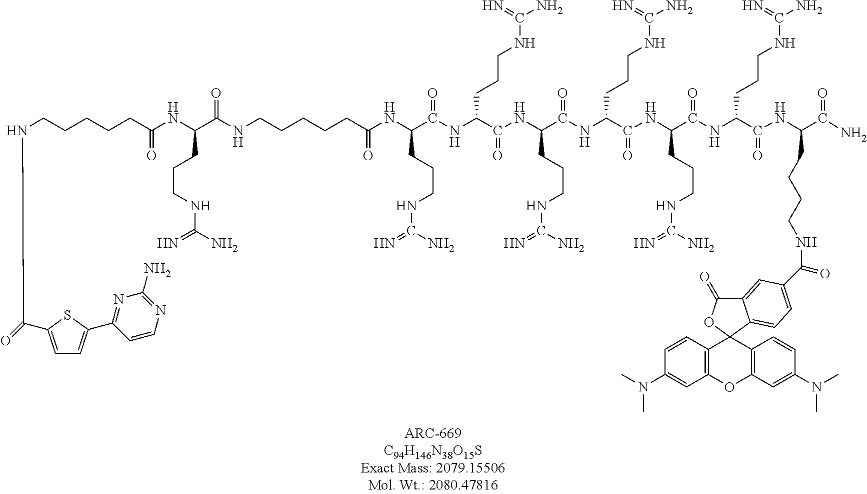 [SMILES: CN(C)c1ccc2c(c1)Oc1cc(N(C)C)ccc1C21OC(=O)c2cc(C(=O)NCCCC[C@@H](NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)[C@@H](CCCNC(=N)N)NC(=O)CCCCCNC(=O)[C@@H](CCCNC(=N)N)NC(=O)CCCCCNC(=O)c3ccc(-c4ccnc(N)n4)s3)C(N)=O)ccc21]

The probe, ARC-669, has high affinity (K_{d}=0.1 nM) towards the catalytic subunit of cAMP-dependent protein kinase (PKAc). It was previously described as a fluorescent probe for fluorescence polarization/anisotropy and fluorescence intensity measurements in steady state conditions [Lavogina et al., Biochim Biophys Acta. 1804 (2010) 1857]. It is displaced from its complex with the kinase by ATP-competitive inhibitor H89, protein substrate competitive inhibitor protein PKI, and bisubstrate inhibitor AdcAhx(D-Arg)₆-NH₂ (ARC-902). Now the compound ARC-669 was developed into a long life-time luminescence probe that binds to several PKs with high affinity (Table 2). The probe can be used as a high-affinity luminescent probe for time-resolved luminescence assays.

When the solution of ARC-669 was illuminated at 337 (50) nm its binding to a PK induced 50-2500-fold increase of the luminescence signal when 50 microsecond delay after the excitation pulse and 100 microsecond measuring window was used for the detection by the intensity of the signal at 590 (40) nm. The luminescence lifetime of the signal was dependent on the origin of the binding PK and it was in the range of 50-300 microseconds.

According to the general structure P-L-F, in the case of the probe ARC-669 P is represented by a aminopyrimidine moiety and a thiophene residue, linker L incorporates a carboxylate group, a 6-aminohexanoic acid residue, a D-arginine residue, a second 6-aminohxanoic acid residue, six D-arginine residues and a D-lysine residue with amidated carboxylate group, F is a 5-carboxytetramethylrhodamine dye connected to the side chain of the lysine residue.

The potential of ARC-669 to inhibit different PKs was tested in a panel of 115 kinases. It was established that ARC-669 binds with high affinity to many PKs (Table 2) and thus it can be used as a luminescent probe with several PKs.

Authors of the present invention have recently shown that highly potent ATP-competitive conjugates of adenosine analogs and arginine-rich peptides are cell membrane permeable [Uri et al., Bioorg. Med. Chem. Lett. 12 (2002) 2117; Enkvist et al., Bioorg. Med. Chem. Lett. 19 (2009) 6098; Vaasa et al., Biochem Biophys Res Commun. 397 (2010) 750], therefore some of the luminescent probes of the present invention are applicable in experiments with live cells, tissues and organisms.

TR-FRET assays are homogeneous and as such they do not require separation steps like chromatography, filtration, centrifugation, precipitation or electrophoresis. Due to the high sensitivity of TR luminescence assay it is well suited for the assay miniaturization, and the assays can be performed with equal success in a cuvette of a fluorescence spectrometer and in low microlitre volumes in wells of 384-well and 1536-well microtiter plates with a fluorescence plate reader with delayed fluorescence measurement possibilities.

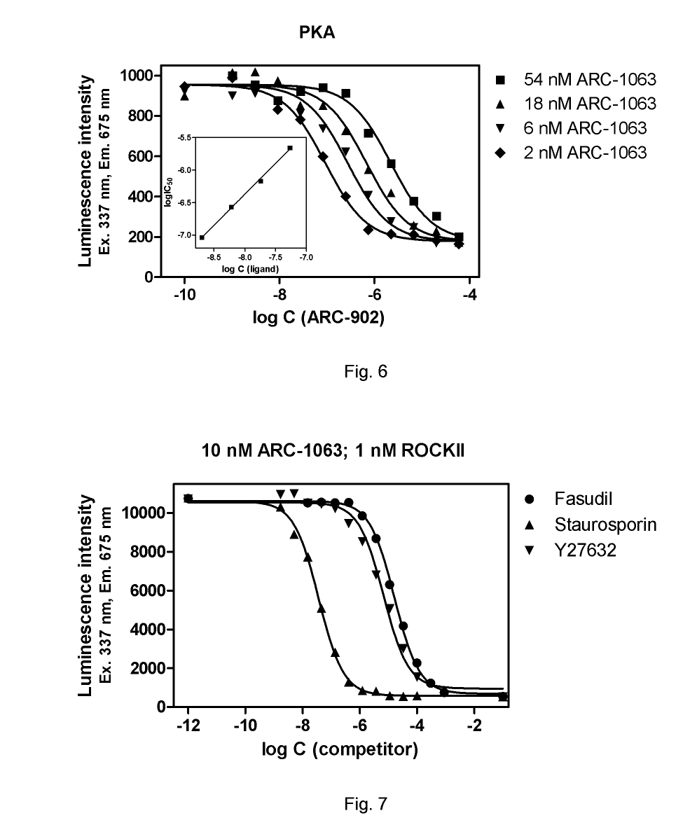

The present invention embodies an assay in which the binding characteristics of the test compounds are determined on the basis of their ability to displace the luminescent probe from its complex with the kinase. This is described in Example 3 and shown on FIG. 6-8.

The high affinity of the luminescent probes of the present invention (K_{d}<1.0 nM) makes it possible to characterize the high-affinity inhibitors with sub-nanomolar and nanomolar affinity. K_{d}-values of the inhibitors could be calculated according to the Cheng-Prusoff equation.

The application of the luminescent probes for the determination of the concentration of the active form of the kinase is another embodiment of the current invention. Determination of the concentration of the special form of a kinase is of paramount importance for many enzyme applications. In kinetic assays the kinase amount in the assay volume is characterized on the activity basis and the molar concentration of the kinase is often not known. Majority of the methods in use for determination of enzyme concentration (Bradford, Lowry, SDS electrophoresis) give the total concentration of the protein (not the concentration of its active form), are inaccurate and need unacceptably large amounts of the protein for analysis.

The application of the photo-luminescent probe of the current invention enables a simple procedure for the determination of the concentration of the active (binding to the luminescent probe) form of the kinase in the sample (Example 2).

It was established that there was a good proportionality between the concentration of the binding protein (as established with the photo-luminescent probe) and the phosphorylation activity of the solution of the PK.

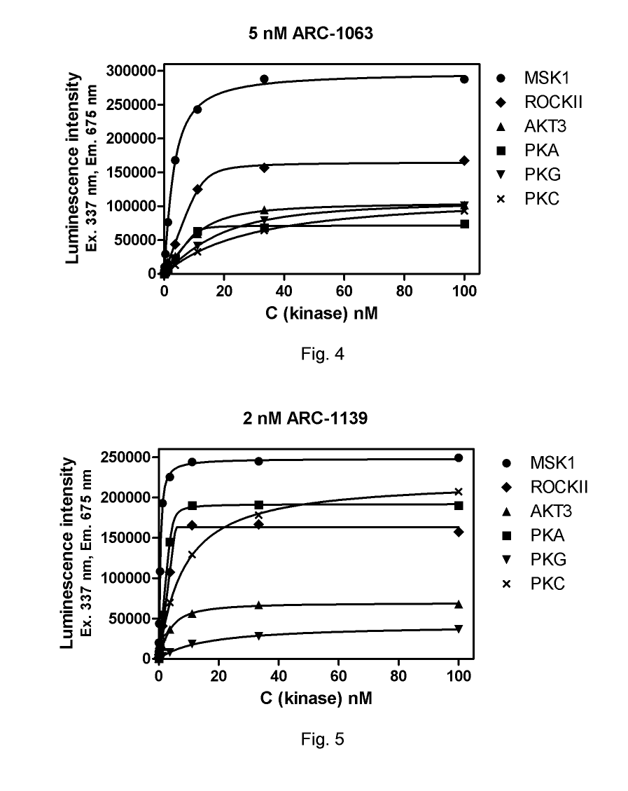

The luminescent probes of the current invention can be used for measurements with several PKs. Titration of probes ARC-1063 and ARC-1139 with many kinases (PKA, MSK1, ROCKII, AKT3, PKG, PKC) resulted in substantial increase of luminescence emission (FIGS. 4 and 5).

A selectivity testing performed (Example 7) for the probe ARC-669 established the inhibitory potency of the compound towards PKs of different groups. Profiling with the kinase panel (Example 7) reveals strong tendency of the compound to inhibit basophilic PKs that points to the active participation of both functionary moieties in the formation of the binary complex with the kinase. In addition to the majority of AGC kinases, many kinases of CAMK and STE groups and some kinases not falling into major groups (classification by [Manning et al., Science 298 (2002) 1912]) were strongly inhibited by the probe. All the inhibited kinases are targets of luminescent probes of the present invention and the probes can be used in analytical methods concerning these kinases. Probes targeted to PKs of other groups can be developed using luminescence donors and acceptors described in the present invention by adding structural elements that support binding of the probes to these kinases. Additionally, the potential targets of the luminescent probes of the present invention are different mutated forms of PKs, truncated forms of kinases inactive states of PKs, and pseudokinases.

In addition to their use in biochemical assays with purified PKs and PKs in complicated biological solutions (cell lysates, tissue extracts, body fluids) the probes of the present invention can be used for monitoring the activity of PKs in living cells (FIGS. 11-13). This enables the application of the probes for monitoring the physiological response of the cells to activation of membrane receptors. Both agonists and antagonists of receptors can be screened and characterized with the application of the probes as far as these receptors activate intracellular signalling cascades involving PKs. Regulation of PKA, e.g., mediated via Gₛ and Gᵢ proteins (FIGS. 11-13), but also isoforms of PKC and Rho-kinase can be monitored with luminescent probes of the present invention in living cells and cell lysates. Thus these probes can be used for screening and characterization of ligands leads to intracellular activation PKs.

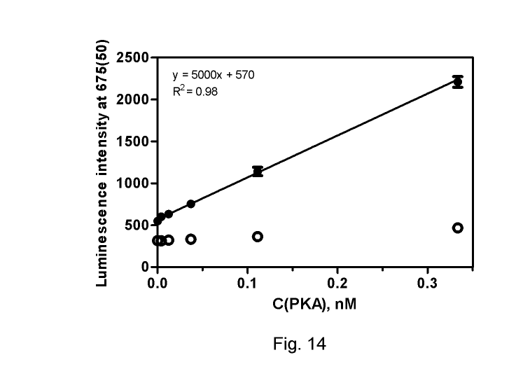

PKs are acknowledged disease biomarkers and changes in the level of their activity in body fluids and tissues signal about diseases like cancer, diabetes, atherosclerosis, etc. The luminescent probes of the current invention can be used for diagnosis of these diseases, for monitoring of the efficiency of the cure of patients, and for supporting drug discovery and development in pharmaceutical industry. Cancer cells leak out PKAc and the higher level of its activity in blood plasma and serum of cancer patients has been reported. [WO/2000/079281] The luminescent probes of the present invention can be used for high throughput analysis of plasma samples for establishment of the level of PKAc activity (FIG. 14 and Example 9). The assay based on the use of luminescent probes of the current invention has very high sensitivity and affords the determination of the concentration of PKA with very low limit of quantification (LoQ=15 pM, 20 microL volume in a well of 384-well microtiter plate). The use of plasma samples makes the diagnosis of cancer minimally invasive for patients and low price and high throughput format of analysis of the samples support the development of analysis methods based on luminescent probes of the present invention for massive analysis of blood samples at hospital laboratories and blood banks for early diagnosis of cancer.

### Experimental Part

### Abbreviations and Definitions

Adc-5′-adenosine carboxylic acid or 1-(6-amino-9H-purin-9-yl)-1-deoxy-β-D-ribofuranuronic acid (CAS 3415-09-6);
Ahx-6-aminohexanoic acid;

### Arg-arginine;

ARC-adenosine-arginine conjugate;
ATP-adenosine-5′-triphosphate;

### Boc-tert-butoxycarbonyl;

cAMP-cyclic adenosine 3′,5′-monophosphate;

### DIPEA-N,N-diisopropylethylamine;

### DMF-dimethylformamide;

DMSO-dimethyl sulfoxide;

### DTT-dithiothreitol

Fmoc-9-fluorenylmethoxycarbonyl;
HOBt-1-hydroxybenzotriazole;
HPLC-high performance liquid chromatography;

### Ip: 2′,3′-O-isopropylidene;

MALDI TOF MS, matrix assisted laser desorption ionisation time-of-flight mass-spectrometry;
NMR-nuclear magnetic resonance;
Pbf-2,2,4,6,7-pentamethyldihydrobenzofuran-5-sulfonyl;
PK-protein kinase;
PKA-cAMP-dependent protein kinase;
PKAc-catalytic subunit of PKA
RP-reverse phase;
TFA-trifluoroacetic acid;
TLC-thin layer chromatography.
ARCs—conjugates of adenosine analogues and arginine-rich peptides are bisubstrate inhibitors of basophilic protein kinases

Bisubstrate inhibitors simultanously associate with two substrate-binding sites of PK and thus combine two inhibitory domains. [Lavogina et al., ChemMedChem 5 (2010) 23] The principle advantage of bisubstrate inhibitors is their ability to generate more interactions with the target enzyme that could result in improved affinity and selectivity of the conjugates, when compared with single-site inhibitors.

Oligopeptide refers a short polymer of amino acids (2-20 amino acids) which are joined together by peptide bonds.

The following terms are defined according to the book Lakowicz Principles of fluorescence spectroscopy, 3rd edition, 2006.

FRET—Förster-type non-radiative dipole-dipole energy transfer [Förster, Ann. Physik. 6 (1948) 55] takes place between two molecules in condition where their energies (emission of donor D with absorption of acceptor A) overlap and they are located at a suitable distance from each other. Typically, a FRET occurs at distance less than 100 Å between the donor and acceptor.

TRF—time-resolved fluorescence. Fluorescence measurements can be broadly classified into two types of measurements: steady-state and time-resolved (TR). Steady-state measurements, the most common type, are those performed with constant illumination and observation. TR measurements are performed after short-pulse excitation of the fluorophore and the emission signal is detected after delay for a certain time period (measurement window). Although measurement of the time-resolved emission requires special instruments, such technology is widely used in biological research because of the increased information available from the data as compared with stationary or steady-state measurements.

Photoluminescence is the emission of light from a substance as a result of absorption of electromagnetic radiation, the emitted light usually has a longer wavelength than the incident radiation. Photoluminescence is formally divided into two categories—fluorescence and phosphorescence—depending on the nature of the excited state. In excited singlet states, the electron in the excited orbital is paired (by opposite spin) to the second electron in the ground-state orbital. Consequently, return to the ground state is spin allowed and occurs rapidly by emission of a photon. The emission rates of fluorescence are typically 10⁸ s⁻¹, so that a typical fluorescence lifetime is in the range of 1-10 ns.

Phosphorescence is emission of light from a triplet excited states, in which the electron in the excited orbital has the same spin orientation as the ground-state electron. Transitions to the ground state are forbidden and the emission rates are slow (10⁻³ to 100 s⁻¹), so that phosphorescence lifetimes are typically milliseconds to seconds.

The luminescence lifetime refers the average amount of time a fluorophore remains in the excited state (following excitation) prior to return to the ground state. Typically, fluorescence lifetimes are near 1-10 ns and a phosphorescence lifetimes are in the range of microseconds to seconds.

### EXAMPLES

### Example 1

### Synthetic Procedures

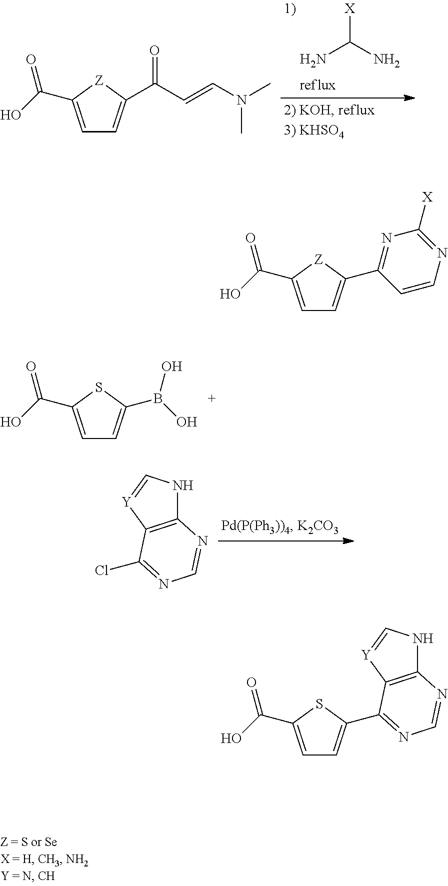

### Synthesis of Adenosine Mimetics

### 5-(3-Dimethylaminoacryloyl)thiophene-2-carboxylic acid methyl ester

2 ml (15 mmol) of N,N-dimethylformamide dimethyl acetal was added to the suspension of 5-acetyl-2-thiophenecarboxylic acid (1013 mg, 5.95 mmol) in DMF (5 ml) and the reaction mixture was refluxed overnight. 100 ml of hexane/tBuOMe (1:1) was added to the cooled reaction mixture and the precipitate was separated by filtration to yield 1140 mg (80%) of the product.

### 5-(2-aminopyrimidin-4-yl)thiophene-2-carboxylic acid

1 (466 mg, 1.95 mmol), guanidine hydrochloride (468 mg, 4.9 mmol) and K 2003 (888 mg, 6.4 mmol) were refluxed in DMF (15 ml) for 3 days. The solvents were removed and the residue was suspended in solution of KOH (0.5 M, 20 ml) and refluxed overnight until clear solution was formed. The product was precipitated with 20% solution of KHSO₄, filtrated, washed and tried to yield the product (360 mg, 83.5%). TLC (DCM/MeOH/AcOH, 9:1:1) R_{f}=0.47.

¹H NMR (CD₃SOCD₃) δ 6.85 (2H, br, NH₂), 7.17 (1H, d, J=5.2 Hz), 7.76 (1H, d, J=4 Hz), 7.92 (1H, d, J=4 Hz), 8.34 (1H, d, J=5.2 Hz), 13.33 (1H, br, COOH).

¹³C NMR (CD₃SOCD₃) δ 104.6, 127.7, 133.7, 136.6, 148.6, 158.0, 159.3, 162.7, 163.5.

### 5-(2-methylpyrimidin-4-yl)thiophene-2-carboxylic acid

The compound was synthesized as 5-(2-aminopyrimidin-4-yl)thiophene-2-carboxylic acid except acetamidine was used instead of guanidine. Yield 85%.

¹H NMR (CD₃SOCD₃) δ 2.63 (3H, s, CH₃), 7.78 (1H, d, J=4.0 Hz), 7.89 (1H, d, J=5.4 Hz), 8.07 (1H, d, J=4.0 Hz), 8.76 (1H, d, J=5.4 Hz), 14.40 (1H, br, COOH).

¹³C NMR (CD₃SOCD₃) δ 25.3, 112.7, 128.2, 133.5, 137.6, 147.2, 157.2, 158.0, 162.2, 167.5.

### 5-(pyrimidin-4-yl)thiophene-2-carboxylic acid

The compound was synthesized as 5-(2-aminopyrimidin-4-yl)thiophene-2-carboxylic acid except formamidine was used instead of guanidine. Yield 74%

¹H NMR (CD₃SOCD₃) δ 7.79 (1H, d, J=4.0 Hz), 8.05-8.15 (2H, m), 8.88 (1H, d, J=5.4 Hz), 9.17 (1H, d, J=1.2 Hz).

¹³C NMR (CD₃SOCD₃) δ 115.7, 128.5, 133.5, 138.1, 146.5, 157.1, 157.8, 158.5, 162.2.

### 5-(2-aminopyrimidin-4-yl)selenophene-2-carboxylic acid

Selenophene-2-carboxylic acid was refluxed overnight in acetic anhydride in the following synthesis was carried out as described for 5-(2-aminopyrimidin-4-yl)thiophene-2-carboxylic acid (except 5-(3-dimethylaminoacryloyl)selenophene-2-carboxylic acid methyl ester did not precipitate from ether/alkane mixture and the solvents were removed before next step). The yield of 5-(2-aminopyrimidin-4-yl)selenophene-2-carboxylic acid from selenophene-2-carboxylic acid was 17%.

¹H NMR (CD₃SOCD₃) δ 6.80 (2H, br, NH₂), 7.16 (1H, d, J=5.1 Hz), 7.97 (1H, d, J=4.2 Hz), 8.12 (1H, d, J=4.2 Hz), 8.32 (1H, d, J=5.1 Hz).

¹³C NMR (CD₃SOCD₃) δ 103.7, 129.6, 136.3, 142.2, 156.0, 159.2, 159.4, 163.6, 164.0.

### 5-(9H-purin-6-yl)thiophene-2-carboxylic acid and 5-(7H-pyrrolo[2,3-d]pyrimidin-4-yl)thiophene-2-carboxylic acid

6-Chloropurine (108 mg, 0.70 mmol) or 6-chloro-7deazapurine (105 mg, 0.68 mmol), 5-carboxythiophene-2-boronic acid (145 mg, 0.84 mmol), K₂ CO₃ (315 mg, 2.3 mmol) and tetrakis(triphenylphosphine)-palladium (120 mg, 0.1 mmol) were stirred overnight at 100° C. under argon. The solvents were removed under vacuum. The residues were dispersed in water and filtrated. The filtrates were acidified with KHSO₄ and the precipitates were collected. The products were used in the solid phase synthesis without further purification.

### Solid-Phase Synthesis of Peptide Conjugates

Peptide fragments were prepared by using traditional Fmoc solid-phase peptide synthesis methods on Rink amide MBHA resin. Protected amino acids (3 equivalents) were dissolved in DMF and activated with HBTU/HOBt (2.94 eq each) in DMF/N-methylmorpholine. Coupling solutions were added to the resin and shaked 40-60 min. The completeness of each step was monitored with Kaiser-test, which was followed by deprotection of Fmoc-group by 20% piperidine solution in DMF (20 min). Fmoc-protected linkers were attached to the peptide part following the same protocol.

1.5 eq of a thiophene or selenophene containing precursor was activated with HOBt/HBTU (1.47 eq each) in DMF/N-methylmorpholine and added to the resin and shaked at least 3 h. Finally the resins were washed 5 times with each solvent (DMF, isopropanol, DCE) and dried. Treatment with TFA/H₂O/triisopropylsilane (90/5/5 by volume) for 2-3 h was used as standard cleavage procedure. The products were purified by reversed-phase HPLC and lyophilized.

Labelling of the ARC-s with Fluorescent Dyes (a Typical Example of the Procedure)

1 μmol of a lysine-containing ARC-type inhibitor and 1.3 μmol N-hydroxysuccinimide esters of the fluorescent dye were dissolved in 50 μl of DMSO and 10 μl triethylamine. The solution was kept 3 h at room temperature and then the solvent were removed in high vacuum. The residue was purified by reversed-phase HPLC and lyophilized to give the desired products in 40-70% of yields.

### Example 2

### Titration of Luminescent Probes with Protein Kinases

Biochemical binding experiments were performed in black, low-volume, 384-well nonbonding surface NBS microplates (Corning) on a PHERAstar plate reader (BMG Labtech) with TRF optic modules [ex. 337(50) nm, em. 675(50) and 620(20) nm] or [ex. 337(50) nm, em. 590(50) and 545(10) nm] using the time-resolved fluorescence mode. The microplates were incubated for 15 min at 30° C. before each measurement. The luminescent probe was excited with a short pulse of flashes (200 flashes per datapoint) at 337 nm, followed by 50 μs delay time and subsequent measurement of the luminescence using a constant acquisition window (150 μs) at the wavelength corresponding to the emission maximum of the conjugated fluorescent dye [590(50)nm or 675(50) nm].

The concentration series of kinases MSK1, ROCKII, AKT3, PKAc, PKG Iα, or PKC δ (3-fold dilutions) was made in the assay buffer (150 mM NaCl, 50 mM Hepes pH=7.5, 5 mM DTT, 7.5 μM BSA) and the fixed concentration of the luminescent probe ARC-1063 or ARC-1139 (5 nM or 2 nM, respectively) was added to each well, incubated and measured as described above. Structures of luminescent probes ARC-1063 and ARC-1139 are listed in Table 1.

Selenophene-based probes (e.g., ARC-1139) gave generally several-fold higher intensity of the emitted signal than the thiophene-based counterparts (e.g., ARC-1063). Minding to the kinase MSK1 led to the strongest signal with both of the luminescent probes, ARC-1063 and ARC-1139. All the tested PKs of the AGC group produced strong TR luminescence signal when bound to the luminescent probes of the current invention (FIGS. 4 and 5).

### Example 3

### Competitive Displacement of a Luminescent Probe by PK Inhibitors

Displacement assay was performed with a concentration series of the competitive compound (3-fold dilutions) in assay buffer (150 mM NaCl, 50 mM Hepes pH=7.5, 5 mM DTT, 7.5 μM BSA) and the fixed concentration of luminescent probe ARC-1063 in complex with a PK was added. The displacement curves were fitted to a sigmoidal dose-response model to obtain IC₅₀ values. K_{d} values were calculated using the Cheng-Prusoff equation (K_{d}=IC₅₀/(1+C_{ARC-Lum}/K_{d ARC-Lum})).

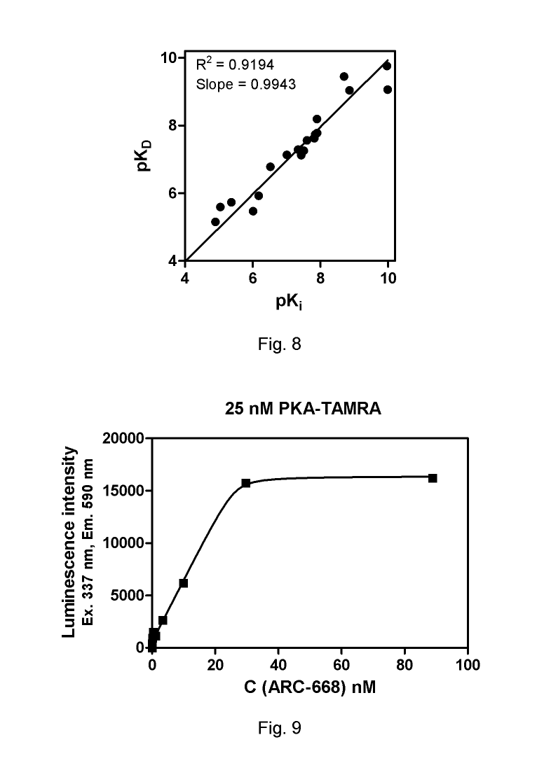

ARC-Lum probes were successfully displaced from kinase complexes by various ATP-competitive, protein substrate competitive and bisubstrate inhibitors (FIGS. 6 and 7). IC₅₀-values of competitive inhibitors were dependent on the concentration of ARC-1063 proving the validity of the Cheng-Prusoff equation in this assay (FIG. 6). Calculated K_{d}-values were in good correlation with Kᵢ-values from enzyme kinetic measurements (FIG. 8). Reliable signals for displacement measurements were obtained even at low, subnanomolar concentration of the PKs tested.

### Example 4

### Enhancement of Luminescence by Intermoleculer Fret (FIG. 2)

A sample of the recombinant kinase PKAc protein was labeled with 5-TAMRA-NHS ester and the labelled protein PKAc-TAMRA was purified by using a Sephadex column. The concentration series of ARC-668 (structure in Table 1) was made in the assay buffer and fixed concentration of PKAc-TAMRA (25 nM) was subsequently added. The luminescence signal was acquired by using the optic module [ex. 337(50) nm, em. 590(50). Intermolecular enhancement of kinase induced long lifetime luminescence by the acceptor was shown to be effective when PKAc was chemically labeled with 5-TAMRA (FIG. 9).

### Example 5

### Measurement of Dissociation Kinetics

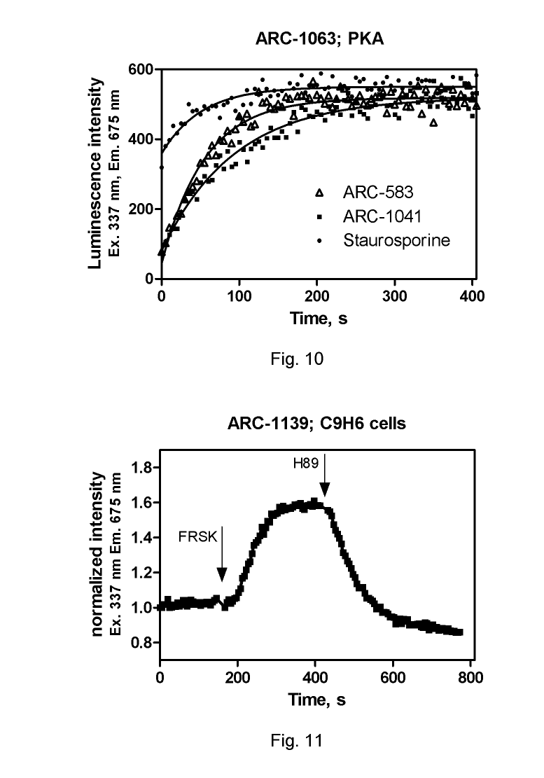

Measurements of dissociation kinetics of non-labeled inhibitors from the inhibitor-kinase complexes (residence time) were carried out by mixing the solution of the examined inhibitor (5-10 nM) with PKAc (10 nM), and incubation of the solution for 15 min at 27° C., followed by addition of a high concentration of the luminescent probe ARC-1063 (final concentration of 100-300 nM), and measurement of the luminescence signal at 5 s time intervals (FIG. 10). The collected data were fitted to the first-order kinetic model yielding the dissociation rate constants for the examined inhibitors.

Dissociation half-lives of PKAc complexes with the tested inhibitors were the following: ARC-1041, 63 s; ARC-583, 43 s; and staurosporine, 12 s (FIG. 10).

### Example 6

### Monitoring of Kinase Activity in Living Cells

CHO, C9H6 and HEK cells were cultured in F12 Nutrient Mixture supplemented with 10% fetal bovine serum, 2 mM L-glutamine, 100 U/ml penicillin and 100 μg/ml streptomycin at 37° C. in humidified atmosphere containing 5% CO₂. In case of C9H6 cells, 300 μg/ml of Zeocine™ and 800 μg/ml of Geneticin® were additionally introduced to the nutrient mixture. Cells were seeded to 96-well plates (for plate reader experiments) and grown to 60%-80% confluency. The medium was replaced with fresh serum-free medium containing the luminescent probe ARC-1139 (10 μM) and incubated for 1 hour at 37° C. Subsequently, the cells were washed three times with PBS and analyzed on a PheraStar platereader with TRF optic modules [ex. 337(50) nm, em. 675(50) and 620(20) nm] using the time-resolved fluorescence measurement mode. Forskolin (final concentration of 25 μM) and H89 (final concentration of 100 μM) were added to the cells during the measurement (after 3 min and 7 min from the start of data acquisition, respectively)

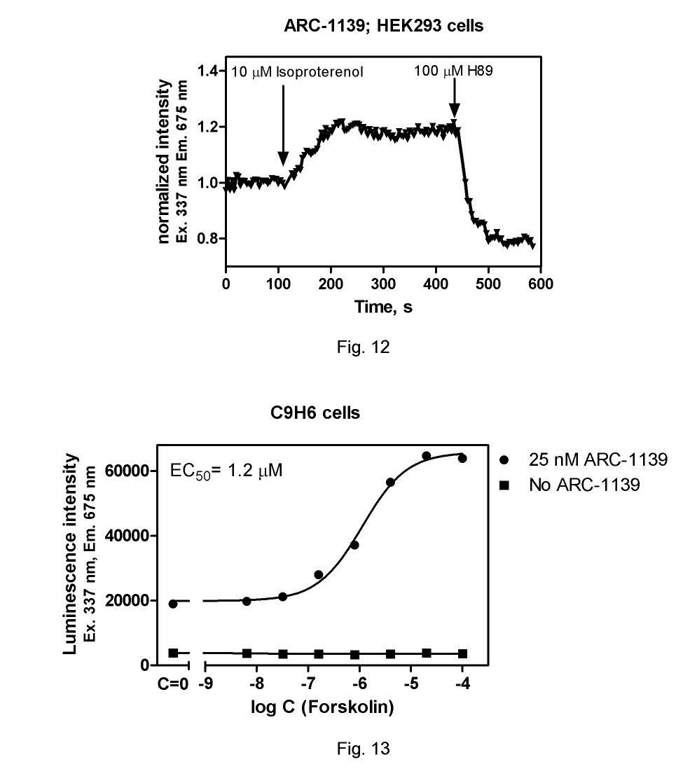

Forskolin caused up to two fold increase of the luminescence from live cells loaded with ARC-1139. The signal decreased after addition of 100 μM H89 that displaces ARC-1139 from its complex with PKAc (FIG. 11). This proves that the luminescent probes can be used for monitoring of kinase activities and effect of the agonists of Gₛ protein-coupled receptors in living cells (FIG. 12).

### Example 7

### PK Selectivity Profiling of a Luminescent Probe

Selectivity testing (Table 2) was performed on the commercial basis at the Division of Signal Transduction Therapy, University of Dundee. ATP concentrations that were used in testing were close to the Kₘ value of the kinase.

Table. 2. Percentages of residual activities of the kinase reaction in the presence of 1 μM ARC-669

TABLE 2

| | | | | | |
|---|---|---|---|---|---|
| MKK1 | 69 | CHK1 | 29 | PAK6 | 35 |
| MKK2 | 68 | CHK2 | 28 | MST2 | 94 |
| MKK6 | 107 | GSK3b | 77 | MST4 | 45 |
| ERK1 | 114 | CDK2-Cyclin A | 78 | GCK | 10 |
| ERK2 | 80 | PLK1 | 101 | MINK1 | 51 |
| JNK1 | 91 | Aurora A | 86 | MEKK1 | 19 |
| JNK2 | 85 | Aurora B | 81 | MLK1 | 60 |
| JNK3 | 77 | LKB1 | 101 | MLK3 | 70 |
| p38a MAPK | 55 | AMPK | 36 | TAO1 | 94 |
| p38b MAPK | 71 | MARK1 | 65 | ASK1 | 74 |
| p38g MAPK | 104 | MARK2 | 77 | TAK1 | 13 |
| p38d MAPK | 104 | MARK3 | 20 | IRAK4 | 82 |
| ERK8 | 23 | MARK4 | 86 | RIPK2 | 47 |
| RSK1 | 2 | BRSK1 | 44 | TTK | 66 |
| RSK2 | 4 | BRSK2 | 77 | MPSK1 | 97 |
| PDK1 | 88 | MELK | 31 | Src | 106 |
| PKBa | 1 | NUAK1 | 12 | Lck | 11 |
| PKBb | 4 | CK1 | 72 | CSK | 51 |
| SGK1 | 8 | CK2 | 7 | YES1 | 64 |
| S6K1 | 4 | DYRK1A | 27 | ABL | 88 |
| PKA | 1 | DYRK2 | 65 | BTK | 36 |
| ROCK 2 | 10 | DYRK3 | 18 | JAK2 | 80 |
| PRK2 | 4 | NEK2a | 55 | SYK | 73 |
| PKCa | 15 | NEK6 | 71 | ZAP70 | 82 |
| PKCγ | 13 | IKKb | 50 | TIE2 | 86 |
| PKCz | 10 | IKKe | 37 | BRK | 100 |
| PKD1 | 37 | TBK1 | 27 | EPH-A2 | 98 |
| STK33 | 76 | PIM1 | 3 | EPH-A4 | 76 |
| MSK1 | 3 | PIM2 | 15 | EPH-B1 | 90 |
| MNK1 | 74 | PIM3 | 7 | EPH-B2 | 96 |
| MNK2 | 76 | SRPK1 | 66 | EPH-B3 | 75 |
| MAPKAP-K2 | 13 | EF2K | 43 | EPH-B4 | 98 |
| MAPKAP-K3 | 31 | HIPK1 | 94 | FGF-R1 | 54 |
| PRAK | 63 | HIPK2 | 63 | HER4 | 82 |
| CAMKKb | 41 | HIPK3 | 97 | IGF-1R | 38 |
| CAMK1 | 3 | CLK2 | 1 | IR | 52 |
| SmMLCK | 46 | PAK2 | 43 | IRR | 32 |
| PHK | 32 | PAK4 | 32 | TrkA | 101 |
| DAPK1 | 20 | PAK5 | 18 | VEG-FR | 25 |

Data of selectivity profiling indicates that the luminescent probe ARC-669 has adequate affinity towards most of the AGC kinases and several PKs from other groups to be used in binding experiments with detection of kinase-induced long lifetime luminescence. Thus the probe can be used for high throughput characterization of inhibitors of many PKs.

### Example 8

### Measurement of Cellular Activation of PKA Via Lysate Analysis

CHO cells stably over-expressing PKAc were grown to 80% confluence on black 96-well micro plates (Corning). Cells were washed once with 300 μL HBSS (Gibco). Thereafter 25 μL of 100 μM IBMX in HBSS containing different concentrations of Forskolin (0-100 μM) were added to the cells and incubated for 10 minutes at 30° C. The cells were lysed by adding 25μL of cell lysis buffer (Invitrogen NP40 cell lysis buffer) containing additionally 1% Triton X, 1× Protease inhibitor cocktail (Sigma-Aldrich Protease inhibitor cocktail for general use), 10 μM PMSF and 0 or 50 nM ARC-1139. The cells were lysed at 30° C. for 10 minutes and the luminescence intensities were measured using a PheraStar plate reader (BMG) with HTRF module TRF excitation filter [(BMG 337), emission at 675(50) nm; 200 flashes, delay 50 μs, integration time 150 μs]. The results were fitted to sigmoidal dose-response model using GraphPad Prism 5 software.

The obtained luminescence intensities were significantly higher when 50 nM ARC-1139 (final concentration in the cell lysate 25 nM) was included in the lysis buffer. The increase of the luminescence signal arises from the formation of the complexes of ARC-1139 and different kinases present in the cell lysate. The luminescence intensity is significantly increased (FIG. 13) as the concentration of Forskolin a cell permeable activator of adenylate cyclase is increased. Adenylate cyclase catalyses the formation of cAMP from ATP and cAMP in turn activates PKA. Thus, luminescent probes of the current invention are cAMP sensors and as such can be used for monitoring the activity of receptor systems. They can be used for screening agonists and antagonists of metabotropic receptors related via G-proteins (e.g., Gₛ and Gᵢ) to the intracellular activation of adenylate cyclises

### Example 9

### Measurements of Kinase Activity in Blood Serum

To human plasma from the local blood bank a concentration series of recombinant PKA was added to the plasma. 20 μL of each plasma sample was added to 384-well micro plate (Corning) already containing 5 μL of 10 nM ARC-1063 (final concentration 2 nM) or 5 μL of 10 nM ARC-1063, 1 μM PKI, 1 mM ATP and 100 mM MgAcetate (final concentrations 5 nM, 200 nM, 200 μM and 20 mM respectively). The samples were incubated at 30° C. for 15 minutes and the luminescence intensities were measured using a PheraStar plate reader (BMG) with HTRF module [TRF excitation filter (BMG 337), emission at 675(50) nm; 200 flashes, delay 100 μs, integration time 100 μs]. All samples were analysed as three replicate points and plotted against the concentration of PKA added to each samples (FIG. 14). The results were fitted to linear regression using GraphPad Prism 5 software.

The obtained luminescence signal is directly proportional to the amount of recombinant PKAc added to the plasma (FIG. 14). Moreover, if PKI, a specific competitive inhibitor of PKAc, is added to the binding assay in the presence of ATP and Mg²⁺ ions the complex of ARC-1063 and PKAc is not formed and only background luminescence (originating from blood components) is detected. Therefore, from the difference of the luminescence signal obtained from the plasma samples in the absence and presence of PKI, the concentration of the endogenous PKAc in plasma can be determined. This sensitive assay has very low limit of quantification (LoQ=15 μM, 20 μL volume in a well of 384-well microtiter plate) that enables the monitoring of PKAc activity in blood samples.

1. A molecular complex comprising an active purine-binding protein, a non-metal long-lifetime luminescence (phosphorescence) donor with heteroaromatic moiety binding to the purine-binding pocket of the protein and a fluorescent luminescence acceptor dye with excitation spectrum, which is at least partially overlapping with the luminescence emission spectrum of the luminescence donor, wherein the molecular complex possesses long lifetime (1-1000 microseconds) photoluminescence properties, if the complex is irradiated at excitation wavelengths of said luminescence donor and the luminescence is measured at emission wavelengths of said fluorescent luminescence acceptor dye.

2. The molecular complex according to claim 1, wherein the luminescence donor with heteroaromatic moiety and fluorescent luminescence acceptor dye are covalently connected by an organic linker.

3. The molecular complex according to claim 1, wherein the fluorescent luminescence acceptor dye is covalently connected to the purine-binding protein.

4. The molecular complex according to claim 1, wherein the luminescence donor contains a thiophene, selenophene or a tellurophene moiety.

5. The molecular complex according to claim 1, wherein the purine-binding protein is a protein kinase.

6. The molecular complex according to claim 5, wherein the compound incorporating the luminescence donor is a bisubstrate inhibitor of a protein kinase.

7. The molecular complex according to claim 1, wherein the purine-binding protein is a protein kinase labelled with a fluorescent luminescence acceptor dye or a protein kinase fused with a fluorescent protein having excitation and emission properties of said fluorescent luminescence acceptor dye.

8. A method for determining of the concentration of the molecular complex of claim 1, wherein the complex is excited with a flash of light in the wavelength range 200-450 nM and the intensity of the emitted light is measured in the wavelength range 450-1000 nm after 0.1-500 microsecond delay time.

9. The method of claim 8, wherein the complex is excited at the wavelengths above 400 nm in the format of multiphoton excitation and the complex emits light in the wavelength range 450-1000 nm.

10. A method for screening and characterization of protein kinase inhibitors based on claim 8, wherein the reduction of the concentration of said molecular complex is determined in the presence of inhibitory compounds.

11. A method for determining binding and dissociation rate constants for inhibitors binding to a purine-binding protein by monitoring the concentration of the molecular complex of claim 1.

12. A binding assay for monitoring the concentration of active protein kinases in living cells based on the determination of the concentration of the molecular complex of claim 1, wherein the luminescence of the cells is measured with a luminescence microscope, a luminescence spectrometer or a luminescence plate reader with pulse excitation and time delayed (time-resolved) luminescence detection possibilities.

13. A method based on the binding assay of claim 10 for in vivo characterization of inhibitors of protein kinases.

14. The method of claim 8 for characterization of agonists and antagonists of Gs and Gi protein-coupled receptors through measuring the change in luminescence intensity caused by the activation of PKA by the second messenger cAMP in living cells.

15. The method of claim 8 for in vivo characterization of agonists and antagonists of metabotropic and ionotropic receptors by measuring the change in the concentration of molecular complex caused by the activation of protein kinases other than PKA.

16. The method of claim 8 for determination of the concentration of active protein kinases in body fluids, cell lysates and tissue extracts.

17. The method of claim 8 for monitoring of receptor activation and cellular concentration of cAMP by analysing cell lysates.

18. The method of claim 8 for determination of the concentration of active protein kinases in body fluids, blood cells, and tissue extracts as biomarkers of diseases.

19. The method of claim 8 for in vivo application for mapping of protein kinase activity in cells, tissues and organisms.

20. The molecular complex according to claim 2, wherein the luminescence donor contains a thiophene, selenophene or a tellurophene moiety.
